(19) United States
(12) Patent Application Publication
(10) Pub. No.: US 2017/0035394 A1
(43) Pub. Date: Feb. 9, 2017

(54) ULTRASONIC DIAGNOSTIC DEVICE

(71) Applicant: HITACHI, LTD., Tokyo (JP)

(72) Inventors: Toshinori MAEDA, Tokyo (JP); Masaru MURASHITA, Tokyo (JP)

(73) Assignee: Hitachi, Ltd., Tokyo (JP)

(21) Appl. No.: 15/038,831

(22) PCT Filed: Nov. 13, 2014
(86) PCT No.: PCT/JP2014/080703

§ 371 Date: May 24, 2016

(30) Foreign Application Priority Data
Nov. 29, 2013 (JP) 2013-247181

(51) Int. Cl. A61B 8/08; G01S 7/52; A61B 8/14

(52) U.S. Cl. CPC A61B 8/5276; A61B 8/0883; A61B 8/14; A61B 8/5207; A61B 8/5246; G01S 7/52077

(57) ABSTRACT

The purpose of the present invention is to use multiresolution decomposition to reduce image sections, such as fogging and stationary artifacts, which appear in an ultrasound image. An image processing unit (20) performs resolution conversion processing on an ultrasound image obtained on the basis of a reception signal, to form a plurality of resolution images having mutually different resolutions, determines, on the basis of the plurality of resolution images, a reduction degree for each section in the image, and forms an ultrasound image in which reduction processing has been performed on each section in the image in accordance with the reduction degrees.

### TECHNICAL FIELD

The present invention relates to an ultrasonic diagnostic device, and particular to image processing of an ultrasonic image.

### BACKGROUND ART

In an ultrasonic image obtained by transmitting and receiving ultrasonic waves, noise referred to as fogging (or artifacts), etc. may appear, in particular near an ultrasonic probe (probe). This fogging is considered to be generated due to, for example, multiple reflection and a side lobe, and causes degradation of the image quality of the ultrasonic image. Therefore, techniques for removing fogging in the ultrasonic image have been proposed.

For example, Patent Document 1 discloses an ultrasonic diagnostic device that suppresses a relatively slow-moving, fixed echo (such as fogging) with a filter for attenuating a particular frequency component using chronologically-received ultrasonic signals.

Patent Document 2 also discloses a method for improving the image quality of an ultrasonic image by applying multiresolution decomposition to the image.

### CITATION LIST

### Patent Documents

Patent Document 1: JP 3683943 B
Patent Document 2: JP 4789854 B

### SUMMARY OF INVENTION

### Technical Problem

However, although, in Patent Document 1, a fixed echo is suppressed when, for example, a relatively low frequency component is attenuated as the particular frequency component, information of a site that is important for diagnosis, such as a relatively slow-moving tissue including the myocardium at the end of ventricular diastole, for example, may also be suppressed. Meanwhile, the technology of multiresolution decomposition disclosed in Patent Document 2 is expected to be applied to ultrasonic images in various ways.

In view of the above-described background, the inventors of the present invention have continued research and development of the technology for reducing an image portion that appears in an ultrasonic image and is referred to as fogging or a stationary artifact. They especially focused on image processing applying multiresolution decomposition.

The present invention has been achieved in the process of that research and development, and the purpose of the present invention is to reduce an image portion of fogging or a stationary artifact that appears in an ultrasonic image, using multiresolution decomposition.

### Solution to Problem

A preferable ultrasonic diagnostic device that serves the above purpose has a probe that transmits and receives ultrasonic waves, a transmitting and receiving section that obtains a received signal from the probe by controlling the probe, a resolution processing section that forms a plurality of resolution images, each having different resolution, by resolution conversion processing of an ultrasonic image obtained based on the received signal, a reduction processing section that determines the degree of reduction in each portion of the image based on the plurality of resolution images, and an image forming section that forms an ultrasonic image subjected to reduction processing according to the degree of reduction in each portion of the image.

In a preferable embodiment, the reduction processing section estimates the degree of structure for each portion in the image based on a difference image between the plurality of resolution images, and determines the degree of reduction for each portion in the image based on the estimation result.

In a preferable embodiment, the reduction processing section estimates the degree of motion for each portion in the image based on images obtained over a plurality of time phases from at least one of the plurality of resolution images, and determines the degree of reduction for each portion in the image based on the estimation result.

In a preferable embodiment, the reduction processing section estimates the degree of structure and the degree of motion for each portion in the image, and determines a subtraction component that determines the degree of reduction for each portion in the image based on the degree of tissue and the degree of motion, and the image forming section forms an ultrasonic image from which the subtraction component is subtracted.

In a preferable embodiment, the reduction processing section subtracts an optimal luminance value determined based on a lowest luminance value in the ultrasonic image from a luminance value of each pixel, thereby generating a subtraction candidate component, and determines the subtraction component based on a subtraction weight and the subtraction candidate component, the subtraction weight being determined according to the degree of structure and the degree of motion.

In a preferable embodiment, the resolution processing section forms, as the plurality of resolution images, at least one high-resolution image and a plurality of low-resolution images; the reduction processing section determines the degree of reduction in each portion in the image based on the plurality of low-resolution images, and forms a low-resolution image component that has been subjected to reduction processing according to the degree of reduction; and the image forming section synthesizes a high-resolution image component obtained from the high-resolution image and the low-resolution image component, thereby forming an ultrasonic image.

### Advantageous Effects of Invention

The present invention reduces an image portion that appears in an ultrasonic image and is referred to as fogging or a stationary artifact, etc., and preferably, removes the image portion completely.

### BRIEF DESCRIPTION OF DRAWINGS

FIG. 1 shows a diagram of an overall structure of an ultrasonic diagnostic device of a preferable embodiment of the present invention.

FIG. 2 shows a diagram of a specific example of an image including fogging.

FIG. 3 shows a diagram for illustrating motion estimation.

FIG. 4 shows a diagram for illustrating motion estimation.

FIG. 5 shows a diagram of a specific example of multiresolution decomposition (myocardium portion).

FIG. 6 shows a diagram for illustrating structure estimation.

FIG. 7 shows a diagram of a specific example of a difference image regarding the myocardium portion.

FIG. 8 shows a diagram of a specific example of multiresolution decomposition.

FIG. 9 shows a diagram of a specific example of a difference image regarding the fogging portion.

FIG. 10 shows a diagram of a specific example of a subtraction candidate component.

FIG. 11 shows a diagram of calculation examples of the weights based on the estimation results.

FIG. 12 shows a diagram of a calculation example of the subtraction weight.

FIG. 13 shows a diagram of a calculation example of a subtraction component.

FIG. 14 shows a diagram of a specific example of fogging removal.

FIG. 15 shows a diagram of an internal structure of an image processing section.

FIG. 16 shows a diagram of an internal structure of a multiresolution decomposition section.

FIG. 17 shows a diagram of an internal structure of a down-sampling section.

FIG. 18 shows a diagram of an internal structure of a high frequency component calculation section.

FIG. 19 shows a diagram of an internal structure of an up-sampling section.

FIG. 20 shows a diagram of an internal structure of a structure calculation section.

FIG. 21 shows a diagram of a specific example of processing in a zero cross removal section.

FIG. 22 shows an internal structure of a data update section.

FIG. 23 shows a diagram of a specific example of processing in the data update section.

FIG. 24 shows an internal structure of a background subtraction section.

FIG. 25 shows a diagram of an internal structure of a weight calculation section.

FIG. 26 shows a diagram of an internal structure of an optimal luminance value estimation section.

FIG. 27 shows a diagram of an internal structure of a subtraction component calculation section.

FIG. 28 shows a diagram of a specific example of processing in a conditioned multiplication section.

FIG. 29 shows a diagram of an internal structure of an image reconstruction section.

FIG. 30 shows a diagram of a variation of the image processing section.

FIG. 31 shows a diagram of an internal structure of the data update section.

FIG. 32 shows a diagram of an internal structure of the background subtraction section.

FIG. 33 shows a diagram of an internal structure of the weight calculation section.

FIG. 34 shows a diagram of another variation of the image processing section.

### DESCRIPTION OF EMBODIMENTS

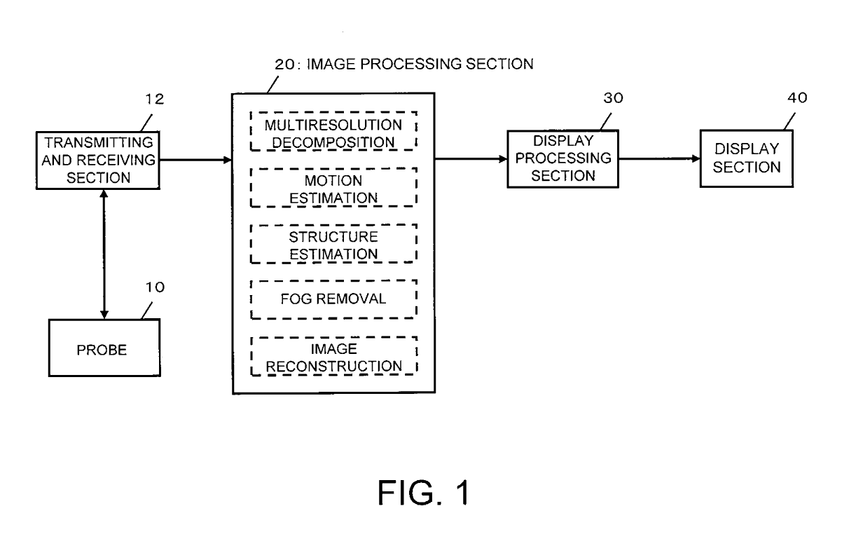

FIG. 1 shows a diagram of an overall structure of an ultrasonic diagnostic device of a preferably embodiment of the present invention. A probe 10 is an ultrasonic probe that transmits and receives ultrasonic waves to/from a region including a diagnostic object, such as, for example, the heart. The probe 10 is provided with a plurality of vibration elements, each transmitting and receiving ultrasonic waves, and the plurality of vibration elements are subjected to transmission control by a transmitting and receiving section 12, to thereby form a transmission beam. The plurality of vibration elements also receive ultrasonic waves from the region including the diagnostic object and output signals thus obtained to the transmitting and receiving section 12, and the transmitting and receiving section 12 forms a receiving beam, to thereby collect echo data along the receiving beam. The probe 10 scans ultrasonic beams (a transmitting beam and a receiving beam) in a two-dimensional plane. Naturally, a three-dimensional probe for scanning ultrasonic waves in three-dimensional space in a three-dimensional way may also be used.

When the ultrasonic beam is scanned in the region including the diagnostic object, and the transmitting and receiving section 12 collects the echo data along the ultrasonic beam; that is, line data, an image processing section 20 forms ultrasonic image data based on the collected line data. The image processing section 20 forms, for example, image data of a B-mode image.

The image processing section 20 forms a plurality of resolution images, each having different resolution, by resolution conversion processing of an ultrasonic image obtained based on a received signal, determines the degree of reduction in portions in the images based on the plurality of resolution images, and forms an ultrasonic image that has been subjected to the reduction processing according to the degree of reduction in the portions in the image. In order to form the ultrasonic image (image data), the image processing section 20 suppresses stationary noise that appears in the ultrasonic image. In particular, noise such as that referred to as fogging (or artifacts) is reduced. In order to reduce the noise such as fogging, the image processing section 20 has the functions of multiresolution decomposition, motion estimation, structure estimation, fogging removal, and image reconstruction. Then, the image processing section 20 forms, for example, a plurality of image data representing the heart, which is the diagnostic object, over a plurality of frames, and outputs the data to a display processing section 30.

The display processing section 30 performs, for example, coordinate conversion processing on the image data obtained from the image processing section 20, the coordinate conversion processing converting the image data from the ultrasonic scanning coordinate system to the image display coordinate system, and further adds a graphic image, for example, as necessary, thereby forming a display image including the ultrasonic image. The display image formed in the display processing section 30 is displayed on a display section 40.

Among the components (function blocks) shown in FIG. 1, the transmitting and receiving section 12, the image processing section 20, and the display processing section 30 can be implemented using hardware, such as a processor and an electronic circuit, and for that implementation, devices such as memories may also be used as necessary. A preferable specific example of the display section 40 is a liquid crystal display, for example.

In addition, except for the probe 10, the components shown in FIG. 1 can also be implemented by a computer, for example. In other words, except for the probe 10, the components shown in FIG. 1 (for example, only the image processing section 20) may be implemented by cooperation of hardware, such as a CPU, a memory, and a hard disk provided in a computer, with software (program) that defines operation of the CPU, etc.

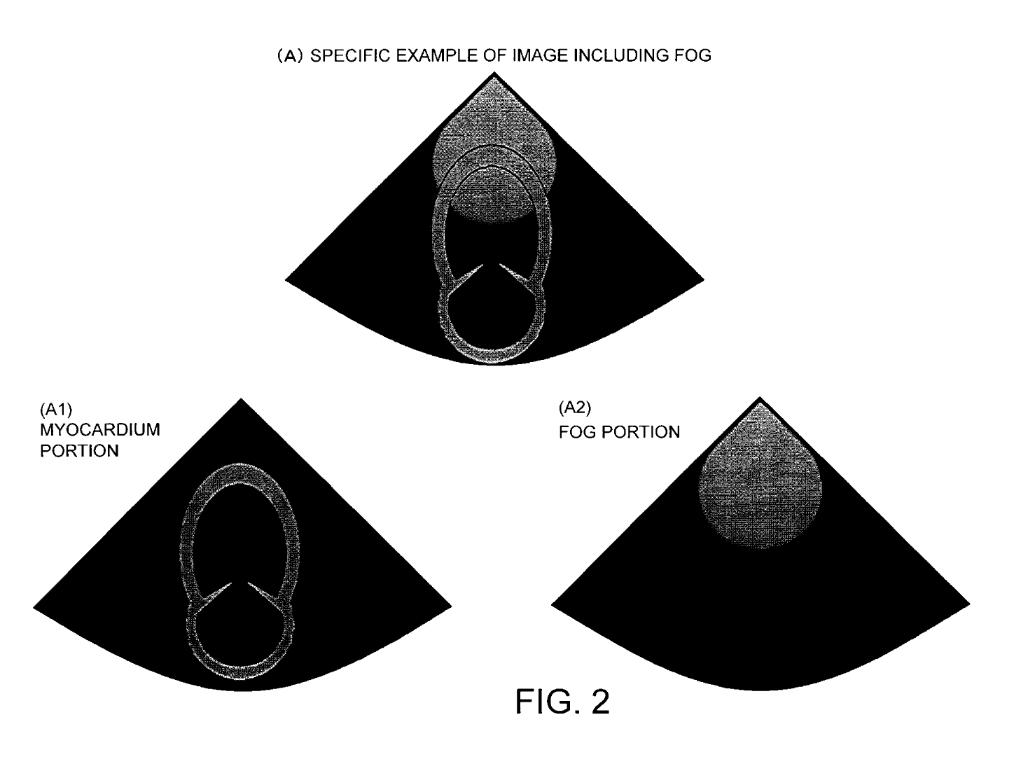

The overall structure of the ultrasonic diagnostic device of FIG. 1 is as described above. Next, the functions implemented by the ultrasonic diagnostic device of FIG. 1 (the present ultrasonic diagnostic device) will be described in detail. In the below description, the components (sections) that are shown in FIG. 1 use the same reference numbers as those in FIG. 1. First, the principle of processing performed in the present ultrasonic diagnostic device (in particular, the image processing section 20) will be described by reference to FIGS. 2 to 14.

FIG. 2 shows a diagram of a specific example of an image including fogging. The image (A) shows a specific example of an ultrasonic image (for example, a B-mode image) of the myocardium including fogging. The myocardium portion and the fogging portion in the image of (A) are shown in (A1) and (A2), respectively. The present ultrasonic diagnostic device identifies, in the ultrasonic image including fogging like (A), the fogging portion shown in (A2), reduces the influence of the identified fogging portion, and preferably removes the fogging portion, thereby forming an ultrasonic image that clearly represents the myocardium portion shown in, for example, (A1). The image processing section 20 of the present ultrasonic diagnostic device identifies the fogging portion in the ultrasonic image by motion estimation and structure estimation.

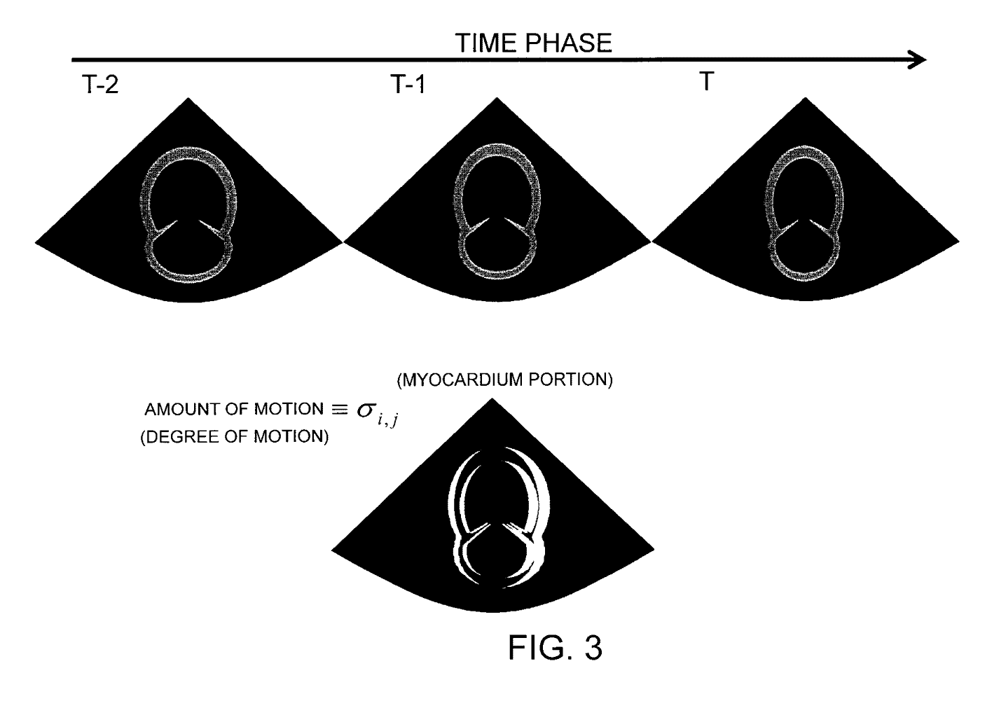

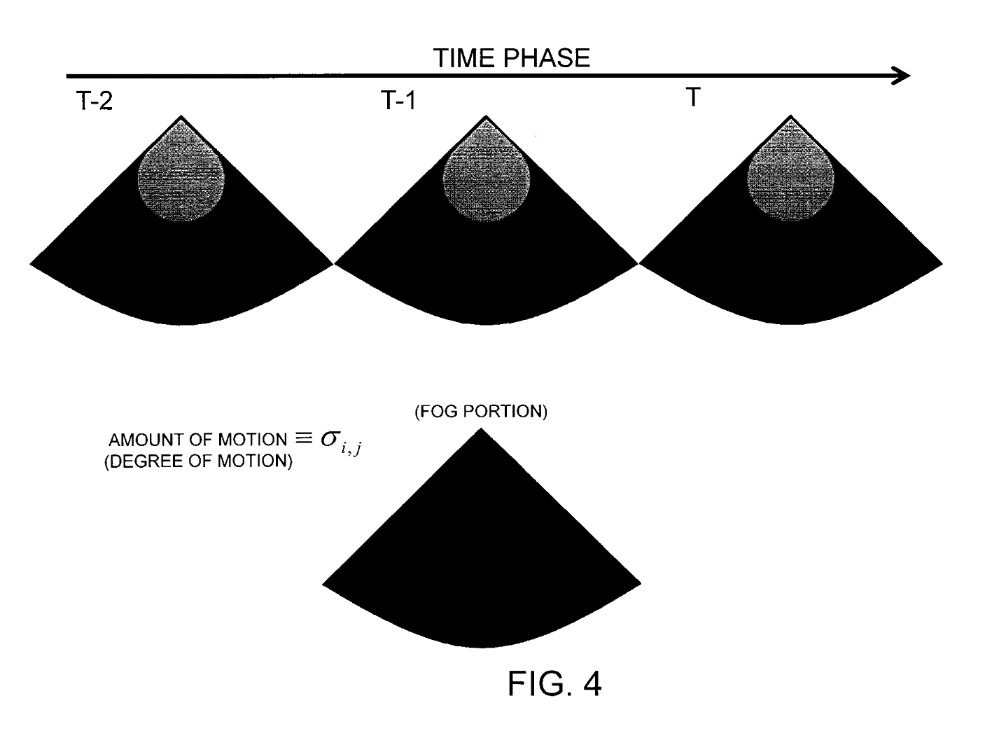

FIG. 3 and FIG. 4 are diagrams for illustrating motion estimation. FIG. 3 and FIG. 4 show specific examples of ultrasonic images obtained over a plurality of time phases (T-2, T-1, T). FIG. 3 shows only the myocardium portion in the ultrasonic images, and FIG. 4 shows only the fogging portion in the ultrasonic images.

The myocardium portion shown in FIG. 3 is in motion in accordance with the diastole and systole motion of the heart. Therefore, a luminance value of a pixel in the myocardium portion in the ultrasonic image changes relatively significantly over the plurality of frames (time phases T2, T-1, T). In contrast, the fogging portion shown in FIG. 4 is almost stationary, and thus, a luminance value of a pixel in the fogging portion in the ultrasonic image hardly changes over the plurality of frames (time phases T2, T-1, T).

Therefore, the image processing section 20, for example, calculates a standard deviation of a luminance value over the plurality of frames (time phases) for each pixel (which is assumed to have the coordinates (i, j)), and use the standard deviation as the index for evaluating the degree of motion (amount of motion). In doing so, it becomes possible to distinguish between the myocardium portion and the fogging portion according to the strength of the degree of motion (the size of the amount of motion).

However, the myocardium portion has a portion having a relatively small degree of motion, for example, in the cardiac wall. Therefore, if, for example, only the degree of motion (amount of motion) is evaluated, the inside of the myocardium portion may not be able to be identified as the myocardium portion.

Therefore, the image processing section 20 of the present ultrasonic diagnostic device further performs structure estimation using multiresolution decomposition, and distinguishes between the myocardium portion and the fogging portion in the ultrasonic image.

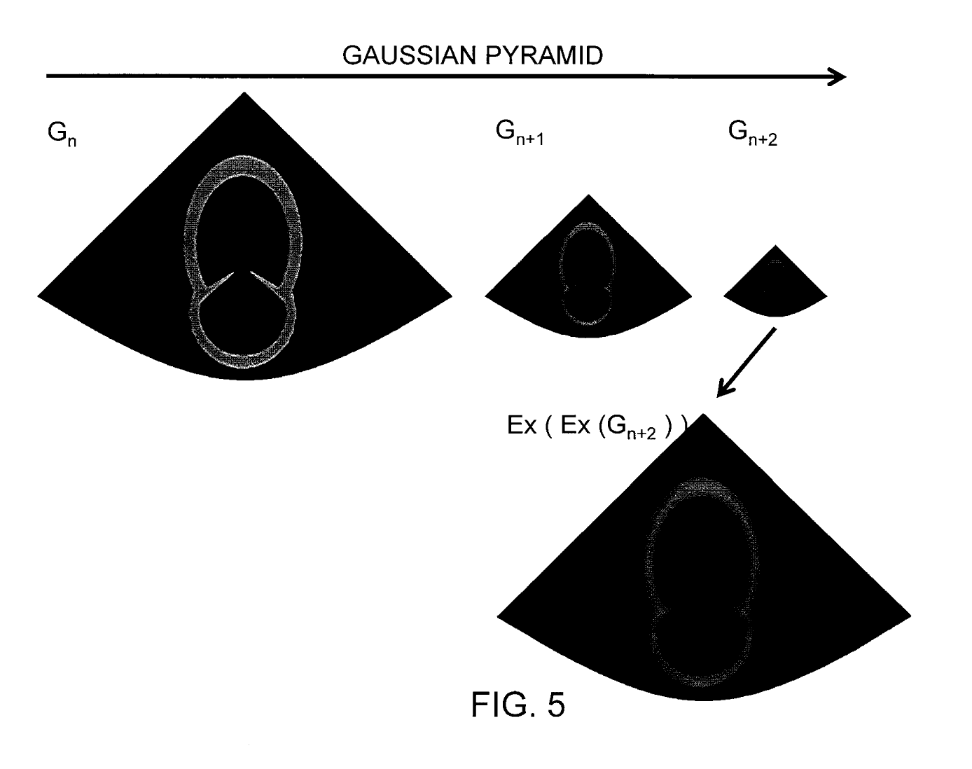

FIG. 5 shows a diagram of a specific example of multiresolution decomposition, and it shows only the myocardium portion in the ultrasonic image. FIG. 5 shows an ultrasonic image Gₙ, a low-resolution image Gₙ₊₁ obtained by performing down-sampling processing on the ultrasonic image Gₙ once, and a low-resolution image Gₙ₊₂ obtained by performing down-sampling processing on the low-resolution ultrasonic image Gₙ₊₁ once. The ultrasonic image Gₙ may be a basic ultrasonic image before resolution conversion or may be a low-resolution image obtained by performing down-sampling processing on the basic ultrasonic image.

Further, it shows a low-resolution image Ex(Ex(Gₙ₊₂)) obtained by performing up-sampling processing on the ultrasonic image Gₙ₊₂ twice. The low-resolution image Ex(Ex(Gₙ₊₂)) has the same resolution as the low-resolution image Gₙ₊₂ and has the same image size as the ultrasonic image Gₙ.

The image processing section 20 compares, for example, the ultrasonic image Gₙ with the low-resolution image Ex(Ex(Gₙ₊₂)) shown in FIG. 5 based on the plurality of resolution images, each corresponding to different resolution, thereby evaluating the degree of structure and performing structure estimation.

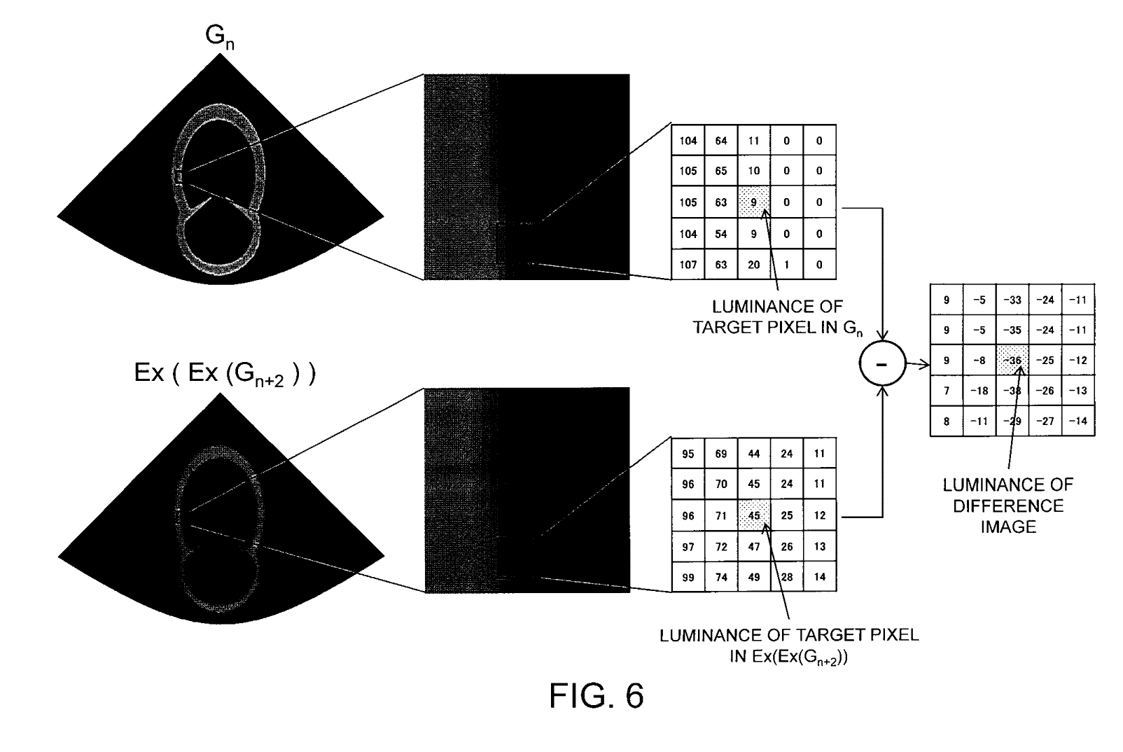

FIG. 6 shows a diagram for illustrating structure estimation. The image processing section 20 forms a difference image between two images; the ultrasonic image Gₙ and the low-resolution image Ex(Ex(Gₙ₊₂)). In other words, in the difference image, a difference in luminance value between corresponding pixels between the two images (pixels having the same coordinates) is adopted as a pixel value of that pixel (a luminance value of the difference).

In the myocardium portion in the ultrasonic image, characteristics of the myocardium tissue (structure) including, for example, minute roughness on the tissue surface or in the tissue are represented. Therefore, for example, if a pixel located on the myocardium surface or in the myocardium is a target pixel, a relatively large luminance difference appears between the target pixel and its surrounding pixels in the ultrasonic image Gₙ, which has relatively high resolution.

In contrast, because the low-resolution image Ex(Ex(Gₙ₊₂)) is a dull (blurred) image compared to the ultrasonic image Gₙ due to the lowered resolution (down-sampling processing), a luminance difference between the target pixel and its surrounding pixels becomes smaller.

Therefore, the larger the luminance difference between the target pixel and the surrounding pixels becomes in the ultrasonic image Gₙ, the greater extent to which the target pixel in the low-resolution image Ex(Ex(Gₙ₊₂)) is changed from the ultrasonic image Gₙ, and the larger a pixel value in the difference image (luminance difference).

Thus, the image processing section 20 determines that the greater the pixel value of the difference image (luminance difference) becomes, the stronger the degree of structure (tissue).

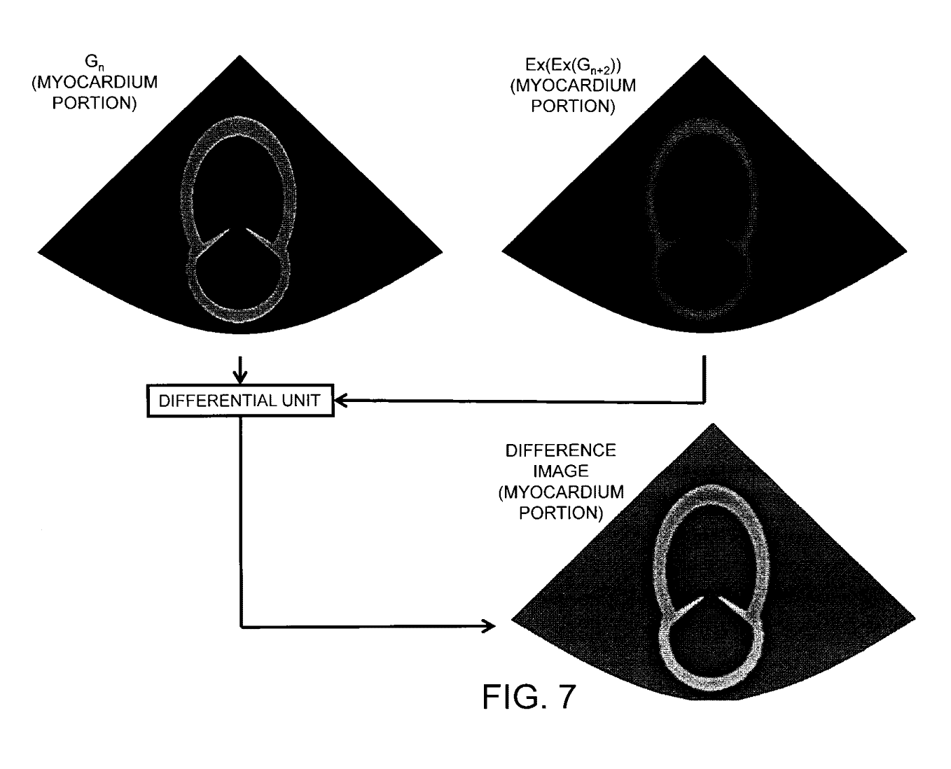

FIG. 7 shows a diagram of a specific example of a difference image regarding the myocardium portion, and it shows the ultrasonic image Gₙ and the low-resolution image Ex(Ex(Gₙ₊₂)) in the myocardium portion, and a specific example of a difference image between these two images.

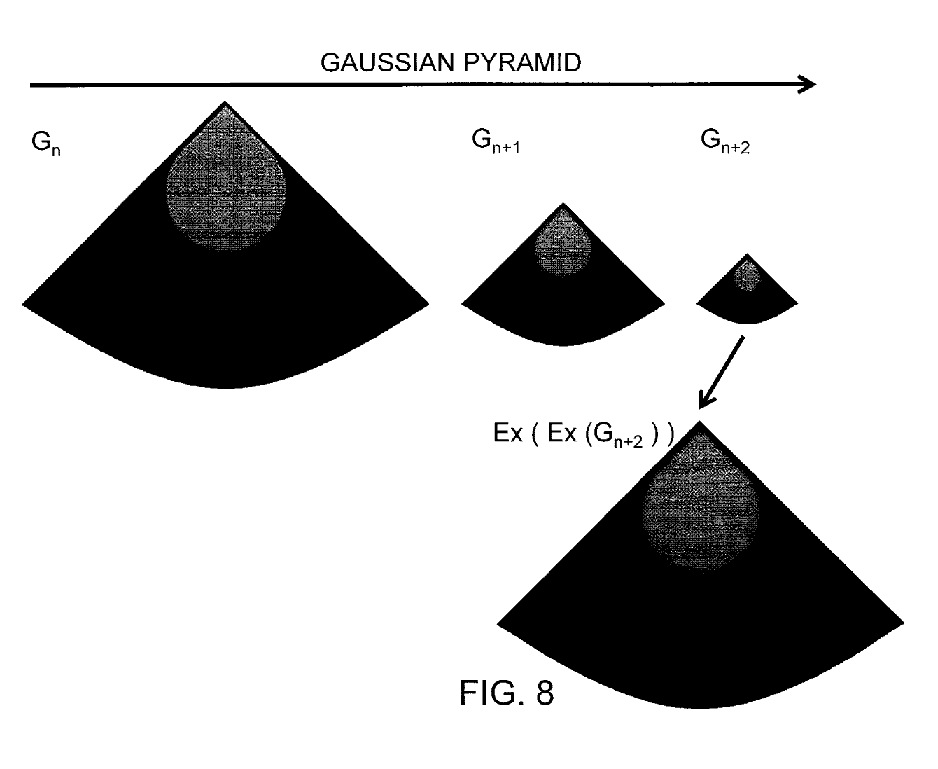

FIG. 8 shows a diagram of a specific example of multiresolution decomposition, and it shows only the fogging portion in the ultrasonic image. FIG. 8 shows the ultrasonic image Gₙ, the low-resolution image Gₙ₊₁ obtained by performing down-sampling processing on the ultrasonic image Gₙ once, and the low-resolution image Gₙ₊₂ obtained by performing down-sampling processing on the low-resolution ultrasonic image Gₙ₊₁ once. The ultrasonic image Gₙ may be a basic ultrasonic image before resolution conversion or may be a low-resolution image obtained by performing down-sampling processing on the basic ultrasonic image.

Further, FIG. 8 shows a low-resolution image Ex(Ex(Gₙ₊₂)) obtained by performing up-sampling processing on the ultrasonic image Gₙ₊₂ twice. The low-resolution image Ex(Ex(Gₙ₊₂)) has the same resolution as the low-resolution image Gₙ₊₂ and has the same image size as the ultrasonic image Gₙ.

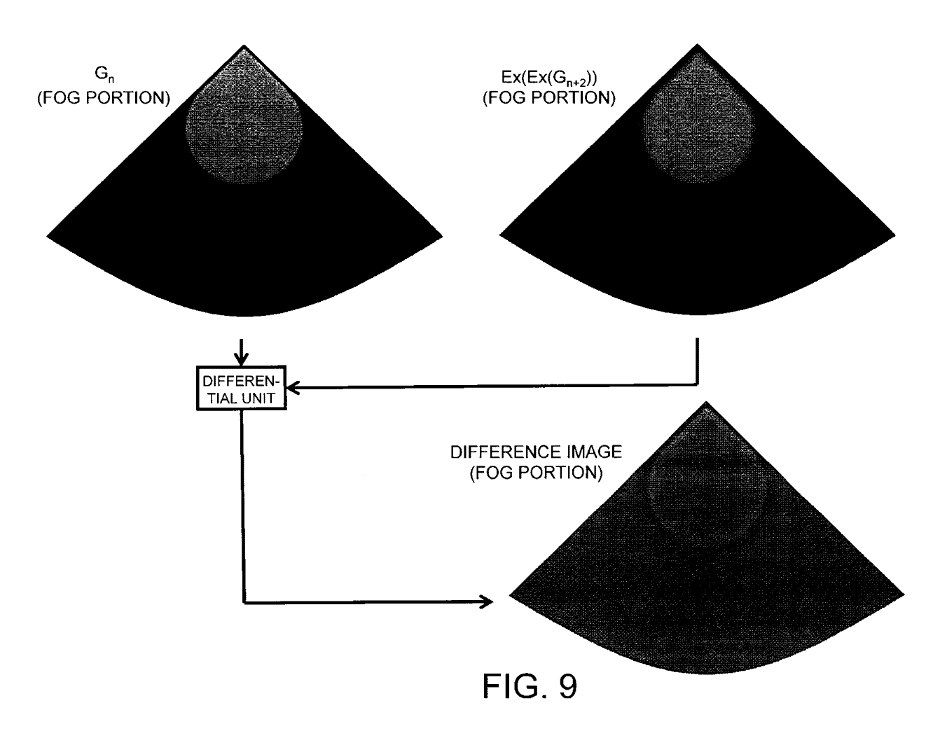

FIG. 9 shows a diagram of a specific example of a difference image regarding the fogging portion, and it shows the ultrasonic image Gₙ and the low-resolution image Ex(Ex(Gₙ₊₂)) in the fogging portion, and a difference image between these two images.

Unlike the myocardium portion (FIG. 7), the fogging portion in the ultrasonic image does not represent the minute roughness of the tissue. Therefore, even if the relatively high-resolution ultrasonic image Gₙ is compared with the low-resolution image Ex(Ex(Gₙ₊₂)) regarding the fogging portion, a large difference does not appear, and the pixel value of the difference image (luminance value) becomes smaller than in the case of the myocardium portion (FIG. 7). Thus, the image processing section 20 determines that the smaller the pixel value of the difference image (luminance difference), the weaker the degree of structure (tissue).

As described in detail below, the image processing section 20 generates a subtraction component for subtracting (removing) the fogging based on the above-described structure estimation and motion estimation.

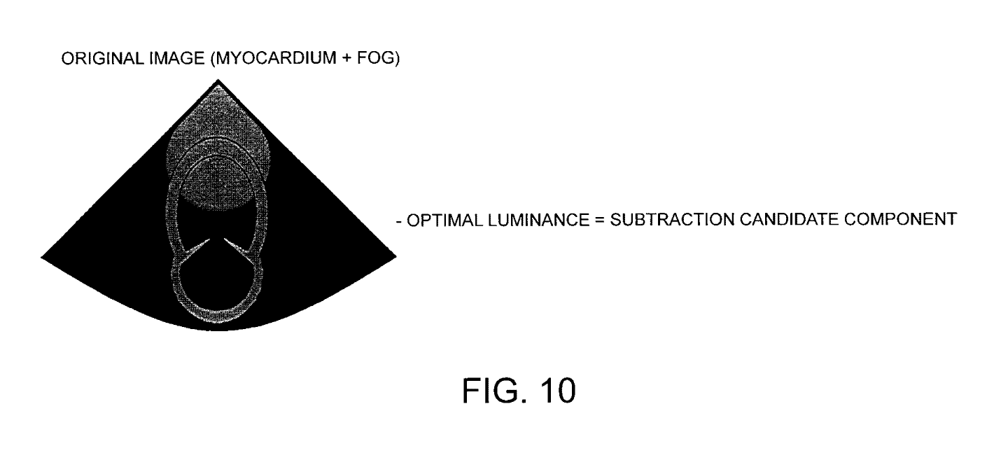

FIG. 10 shows a diagram of a specific example of a subtraction candidate component. Upon generation of the subtraction component, the image processing section 20 generates a subtraction candidate component which is a range that may be subtracted, in order to avoid excessive reduction (excessive shaving) of image information.

For, example, as shown in FIG. 10 by subtracting, from the luminance value of each pixel, an optimal luminance value (optimal luminance) determined based on the lowest luminance value among all the pixels (lowest luminance), the subtraction candidate component is generated.

Then, the image processing section 20 calculates the weight in subtraction from the structure estimation result and the motion estimation result.

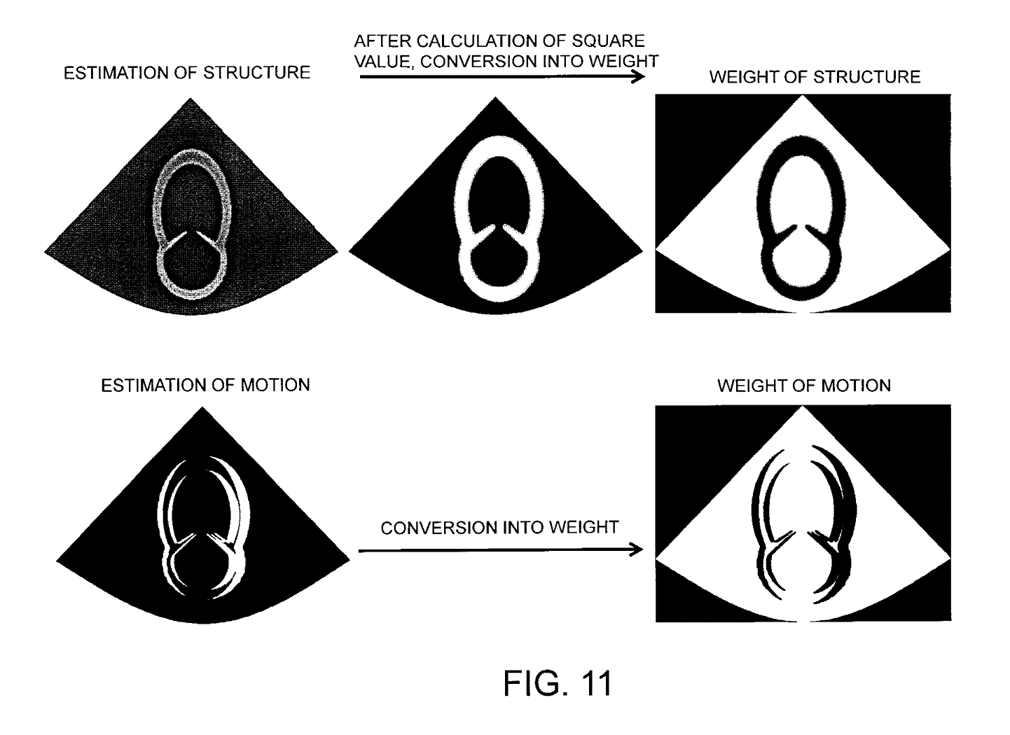

FIG. 11 shows a diagram of calculation examples of the weights based on the estimation results. The image processing section 20 calculates the weight of structure based on the result obtained by structure estimation. The image processing section 20, for example, squares the luminance value of each pixel in the difference image (FIG. 7 and FIG. 9), to thereby calculate the weight of structure for each pixel. The image processing section 20 also calculates the weight of motion based on the results obtained by motion estimation. The image processing section 20, for example, calculates the weight of motion for each pixel based on the amount of motion for each pixel (FIG. 3 and FIG. 4) obtained by the motion estimation. The image processing section 20 then calculates the subtraction weight based on the weight of structure and the weight of motion.

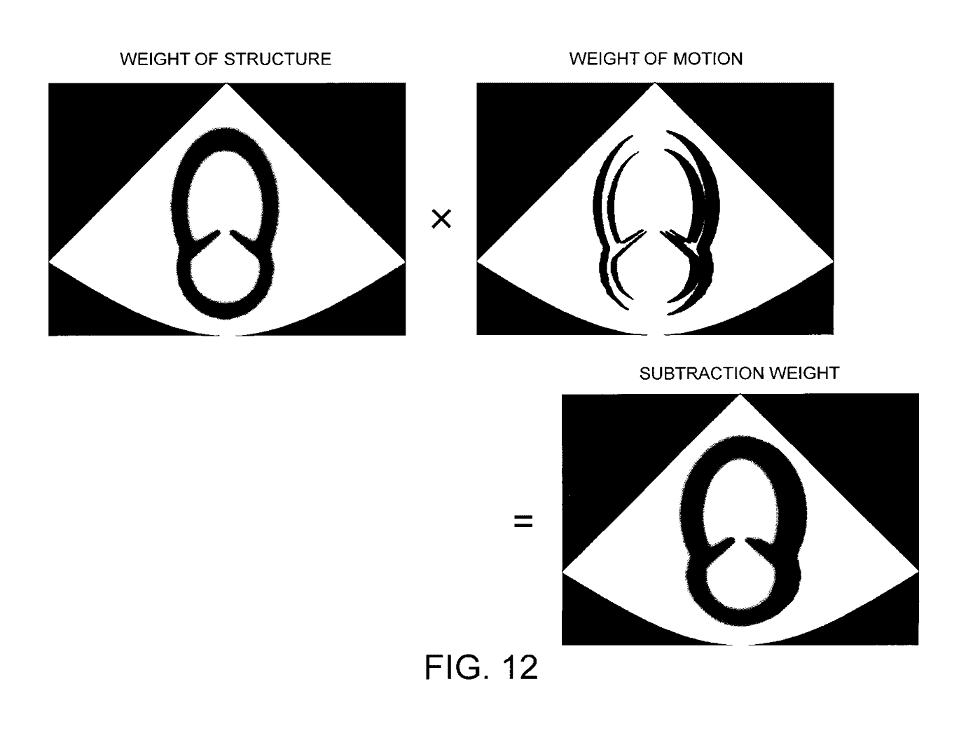

FIG. 12 shows a diagram of a calculation example of the subtraction weight. The image processing section 20 multiplies, for example, the weight of structure and the weight of motion; that is, the weight of structure and the weight of motion for each pixel, thereby calculating the subtraction weight.

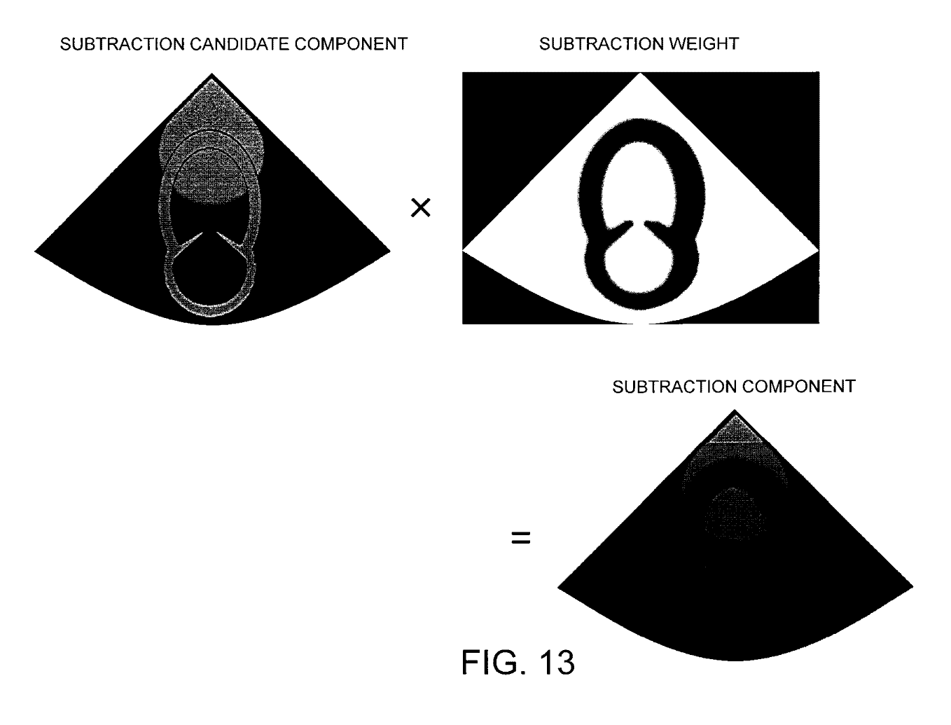

FIG. 13 shows a diagram of a calculation example of a subtraction component. The image processing section 20 multiplies, for example, the subtraction candidate component (FIG. 10) and the subtraction weight (FIG. 12); that is, the subtraction candidate component and the subtraction weight for each pixel, thereby calculating the subtraction weight.

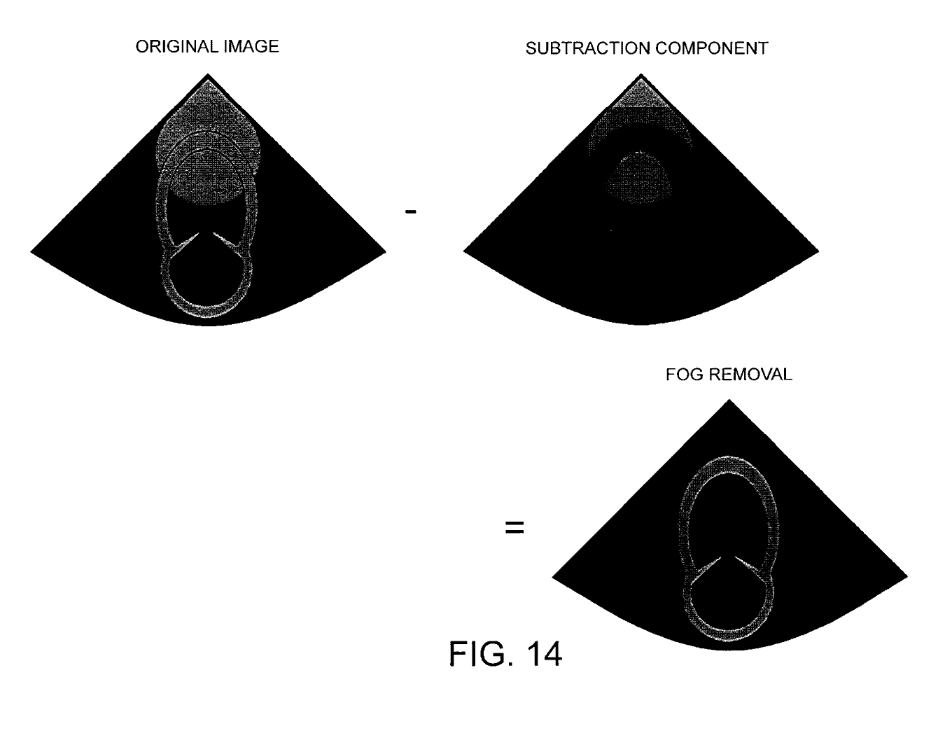

FIG. 14 shows a diagram of a specific example of fogging removal. The image processing section 20 subtracts the subtraction component (FIG. 13) from the original image (FIG. 10) including the myocardium portion and the fogging portion, and more specifically, the image processing section 20 subtracts, for each pixel, the subtraction component from the pixel value of the original image, thereby forming an ultrasonic image where the fogging is reduced, and more preferably, the fogging is removed.

With the above-described processing, the fogging portion having the small degree of structure and the small degree of motion is reduced or removed from the original image including the myocardium portion and the fogging portion so as to maintain the myocardium portion as much as possible and, more preferably, to maintain the myocardium portion completely. Next, a specific structure example of the image processing section 20 for implementing the above-described processing will be described.

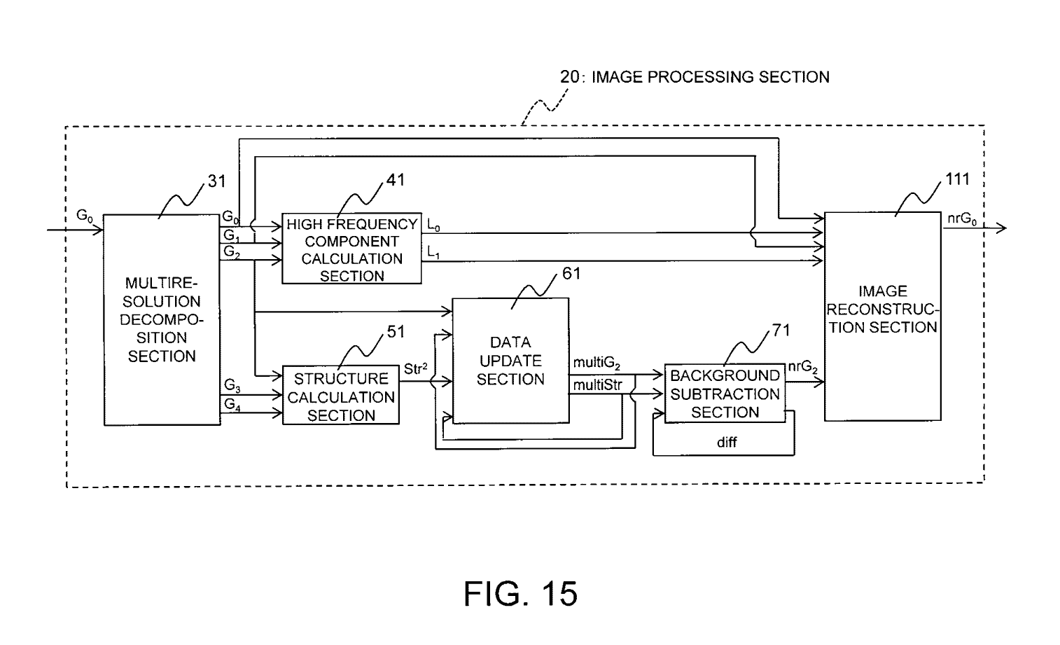

FIG. 15 shows a diagram of an internal structure of the image processing section 20. The image processing section 20 has a multiresolution decomposition section 31, a high frequency component calculation section 41, a structure calculation section 51, a data update section 61, a background subtraction section 71, and an image reconstruction section 111. The line data obtained by the image processing section 20 from the transmitting and receiving section 12; that is, the image data G₀ of the diagnostic image (for example, the original image of FIG. 10) is first subjected to processing in the multiresolution decomposition section 31.

The multiresolution decomposition section 31 creates a Gaussian pyramid of the input diagnostic image. The input diagnostic image is assumed to be G₀, and data of each layer generated in multiresolution decomposition section 31 is assumed to be a Gₙ component (where n is an integer greater than or equal to 0).

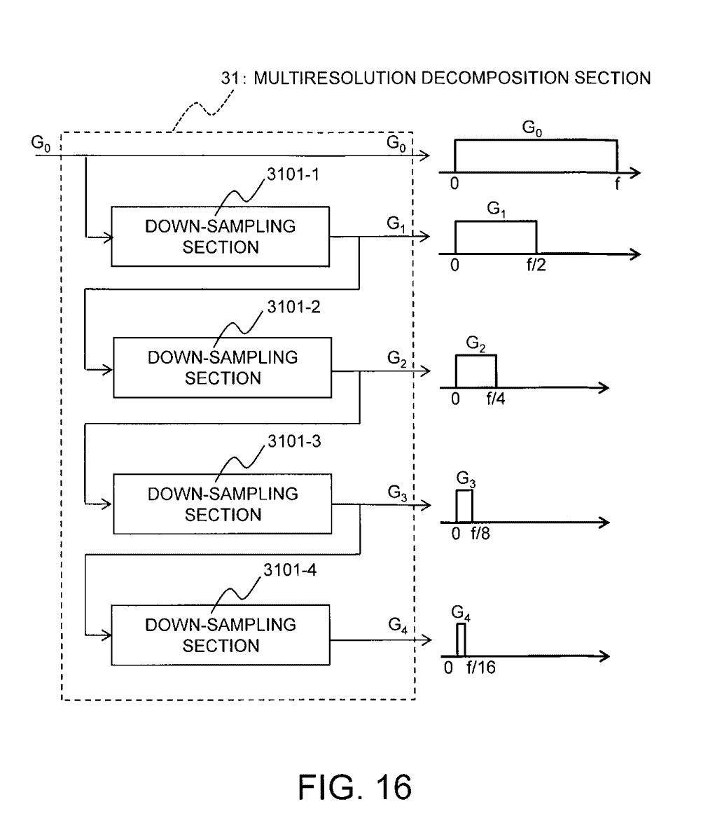

FIG. 16 shows a diagram of an internal structure of the multiresolution decomposition section 31 (FIG. 15). The multiresolution decomposition section 31 has the structure as shown in the figure, and the input Gₙ components are input to down-sampling sections 3101-1, 3101-2, 3101-3, and 3101-4 and are subjected to down-sampling processing by a method as described below.

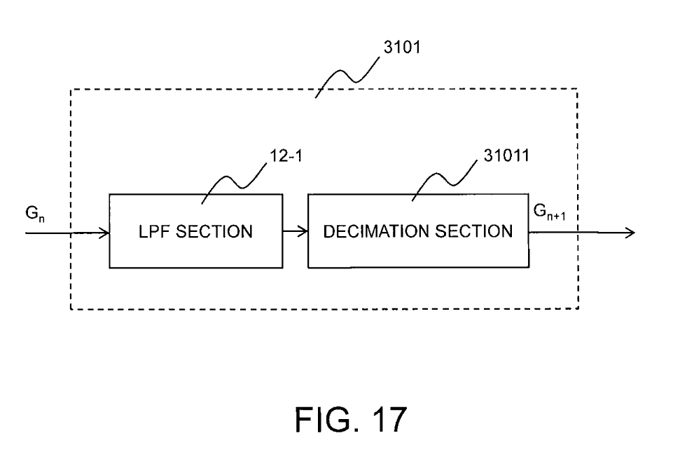

FIG. 17 is a diagram of an internal structure of the down-sampling section 3101 (FIG. 16). The down-sampling section 3101 has a structure as shown in the figure. A low-pass filter (LPF) section 12-1 applies a two-dimensional low-pass filter (LPF) to the Gₙ component, and a decimation section 31011 thins down the data output from the LPF section 12-1 and applies decimation processing to the result, thereby generating a Gₙ₊₁ component in which the sample density and the resolution are reduced.

Thus, the Gₙ component generated in the multiresolution decomposition section 31 in FIG. 16 has multi-resolution expression where the sample density and the resolution differ from the G₀ component. For example, specific examples of frequency bands of the diagnostic image G₀ which is the original image and the G₁, G₂, G₃, and G₄ components obtained from G₀ by down-sampling are as shown in FIG. 16.

In the specific example shown in FIG. 16, when G₀ has a frequency band of 0 to f, G₁ has a frequency band of 0 to f/2; G₂ has a frequency band of 0 to f/4; G₃ has a frequency band of 0 to f/8; and G₄ has a frequency band of 0 to f/16. As a variation, down-sampling may also be performed such that, when G₀ has a frequency band of 0 to f, G₁ has a frequency band of 0 to 4f/5; G₂ has a frequency band of 0 to 3f/5; G₃ has a frequency band of 0 to 2f/5; and G₄ has a frequency band of 0 to f/5.

Although, in the specific example shown in FIG. 16, the highest layer of multi-resolution expression is assumed to be 4 (n=4), this specific example is merely an example, and multiresolution decomposition may be performed within a range between layer 0 and layer n (n≧1).

Further, although, in the above specific example, the decimation processing is performed after the two-dimensional low-pass filter is applied in the down-sampling section 3101 (FIG. 17), this is not limiting, and decimation may be performed after a one-dimensional low-pass filter is applied in each direction, or the decimation processing may be performed while the one-dimensional low-pass filter is applied.

Further, in the low-pass filter (LPF) described below, the two-dimensional low-pass filter may be applied, or the one-dimensional low-pass filter may be applied in each dimension. Further, although in the above specific example, the structure in which the Gaussian pyramid processing is performed has been described as an example of the multiresolution decomposition section, the structure may be changed to a structure in which multiresolution decomposition is performed using, for example, discrete wavelet transformation, Gabor transformation, or a band-pass filter in the frequency domain.

Referring again to FIG. 15, the Gₙ component obtained in the multiresolution decomposition section 31 is input to the high frequency component calculation section 41, the structure calculation section 51, the data update section 61, and the image reconstruction section 111. At this time, the blocks may have, as an input, only necessary data for each of them, or they may share all data. In addition, when the data is input to each of the blocks, the data may be subjected to any filtering processing and the like. The high frequency component calculation section 41 generates a Laplacian pyramid used in image reconstruction.

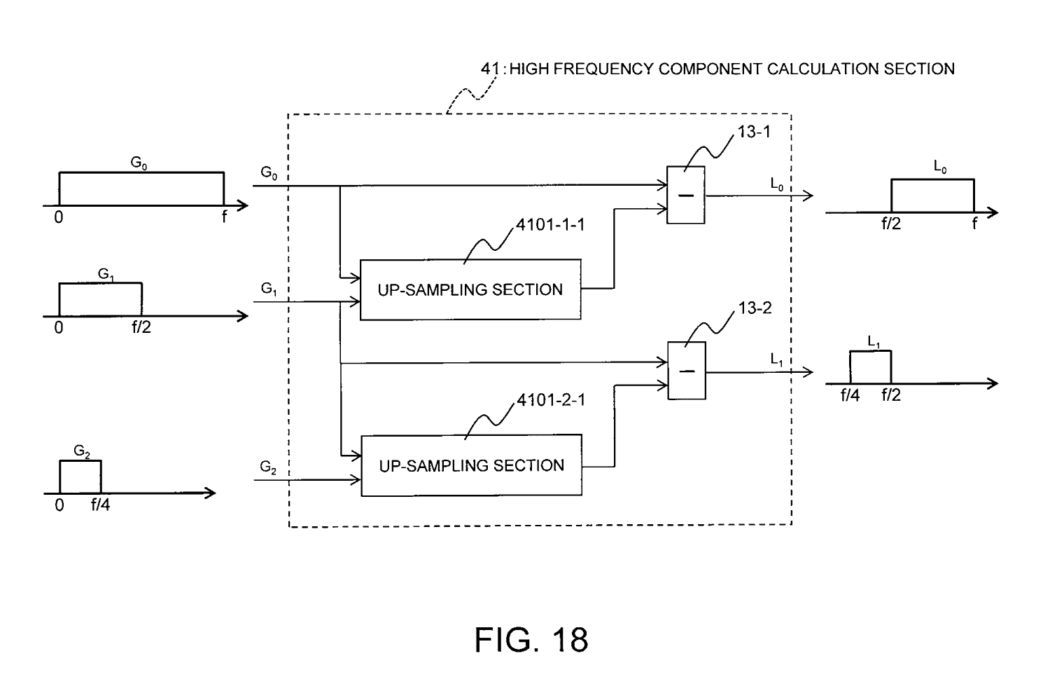

FIG. 18 shows a diagram of an internal structure of the high frequency component calculation section 41 (FIG. 15). The high frequency component calculation section 41 has the structure as shown in the figure, and the input Gₙ₊₁ component is input to up-sampling sections 4101-1-1 and 4101-2-1 and is subjected to up-sampling processing by a method as described below. The components subjected to up-sampling are input to subtracters 13-1 and 13-2 along with the Gₙ components and are subjected to difference processing, thereby calculating an Lₙ components which are high frequency components.

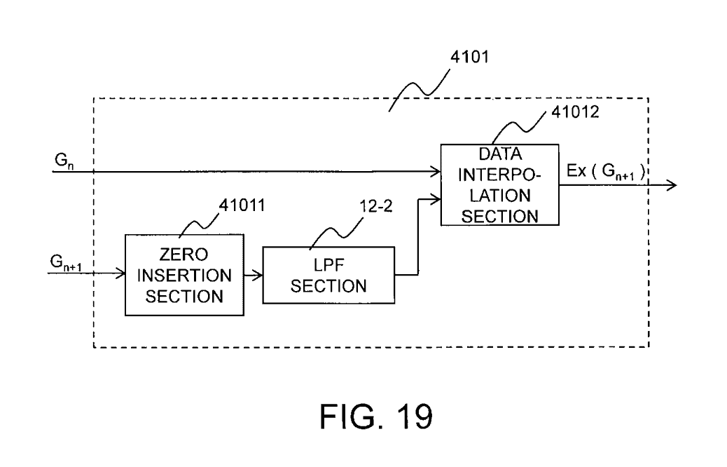

FIG. 19 shows a diagram of an internal structure of an up-sampling section 4101 (FIG. 18). The up-sampling section 4101 has the structure as shown in the figure. A zero insertion section 41011 applies zero insertion processing of inserting zero into the Gₙ₊₁ component at intervals where data are skipped, and the Gₙ₊₁ component into which zero has been inserted is subjected to the low-pass filter (LPF) in the LPF section 12-2. In a data interpolation section 41012, the component subjected to the above-described processing is subjected to interpolation so as to have a size (image size) equal to that of the Gₙ component, thereby obtaining the up-sampled EX(Gₙ₊₁) component.

Thus, the data of the layers created in the high frequency component calculation section 41 in FIG. 18 are referred to as Lₙ components (n≧0). The Lₙ component has edge information which differs for each layer.

FIG. 18 shows a specific example of a frequency band of each component. Assuming that the frequency band of the diagnostic image G₀, which is the original image, is 0 to f; that the frequency band of the diagnostic image G₁ is 0 to f/2; and that the frequency band of G₂ is 0 to f/4 (see FIG. 16), because the L₀ component is obtained based on a difference between G₀ and G₁, the frequency band becomes f/2 to f, and because the L₁ component is obtained based on a difference between G₁ and G₂, the frequency band becomes f/4 to f/2.

Although, in the above-described specific example, the G₀ component to the G₂ component have been input to the high frequency component calculation section 41 to obtain the L₀ component and the L₁ component, this specific example is not limiting, and, for example, Gₙ components of more layers may also be input to obtain more Lₙ components.

Further, although, in the above-described specific example, the structure in which the Laplacian pyramid processing is performed as an example of high frequency component calculation has been indicated, it may be changed to a structure in which the high frequency component is calculated using, for example, discrete wavelet transformation, Gabor transformation, or a band-pass filter in the frequency domain.

Referring to FIG. 15 again, the L₀ component and the L₁ component obtained in the high frequency component calculation section 41 are input to the image reconstruction section 111. In addition, the structure calculation section 51 calculates a structure estimation value Str² used in estimation of the structural strength (structure estimation).

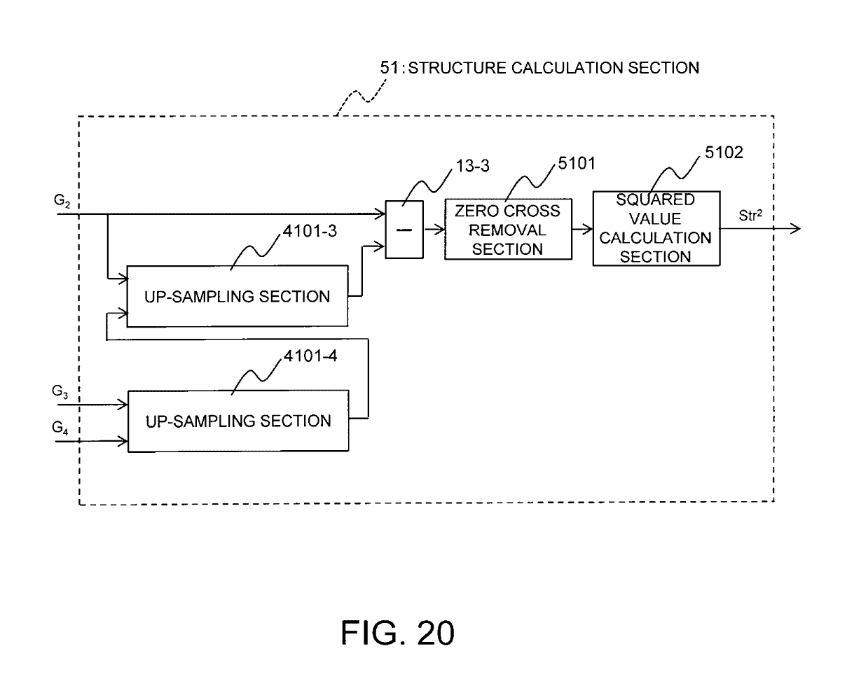

FIG. 20 shows a diagram of an internal structure of the structure calculation section 51 (FIG. 15). The structure calculation section 51 has the structure as shown in the figure. The input Gₙ₊₂ component is input to an up-sampling section 4101-4 to thereby be subjected to the up-sampling processing, and the up-sampled component is subjected to the up-sampling processing again in an up-sampling section 4101-3 and then input to a subtracter 13-3 along with the Gₙ component. A difference value calculated in the subtracter 13-3 is input to a zero cross removal section 5101 and is subjected to a below-described zero cross removal processing, and then the result is input to a square value calculation section 5102 to thereby calculate the structure estimation value Str². The structure estimation value Str² generated in the structure calculation section 51 is data having information of the structure strength.

Although, in the above-described specific example, assuming that n=2, the structure estimation value Str² is obtained by inputting the G₂ component to the G₄ component to the structure calculation section 51, this specific example is not limiting, and, for example, at least two components of the Gₙ components may be input to obtain the structure estimation value Str².

Further, although, in the above-described specific example, the structure estimation value Str² is obtained by obtaining the difference between the G₂ component and the component obtained by up-sampling the G₄ component twice, this specific example is not limiting, and the difference may be obtained using consecutive layers or more separate layers. Further, although, in the above-described specific example, the difference between the G₂ component and the component obtained by up-sampling the G₄ component twice has been calculated, the final structure estimation value Str² may be calculated by calculating another component, such as, for example, a difference between the G₁ component and a component obtained by up-sampling the G₃ component twice, and considering structure estimation values respectively calculated from the two of the difference obtained from the G₂ component and G₄ component and the difference obtained from the G₁ component and the G₃ component (two differences).

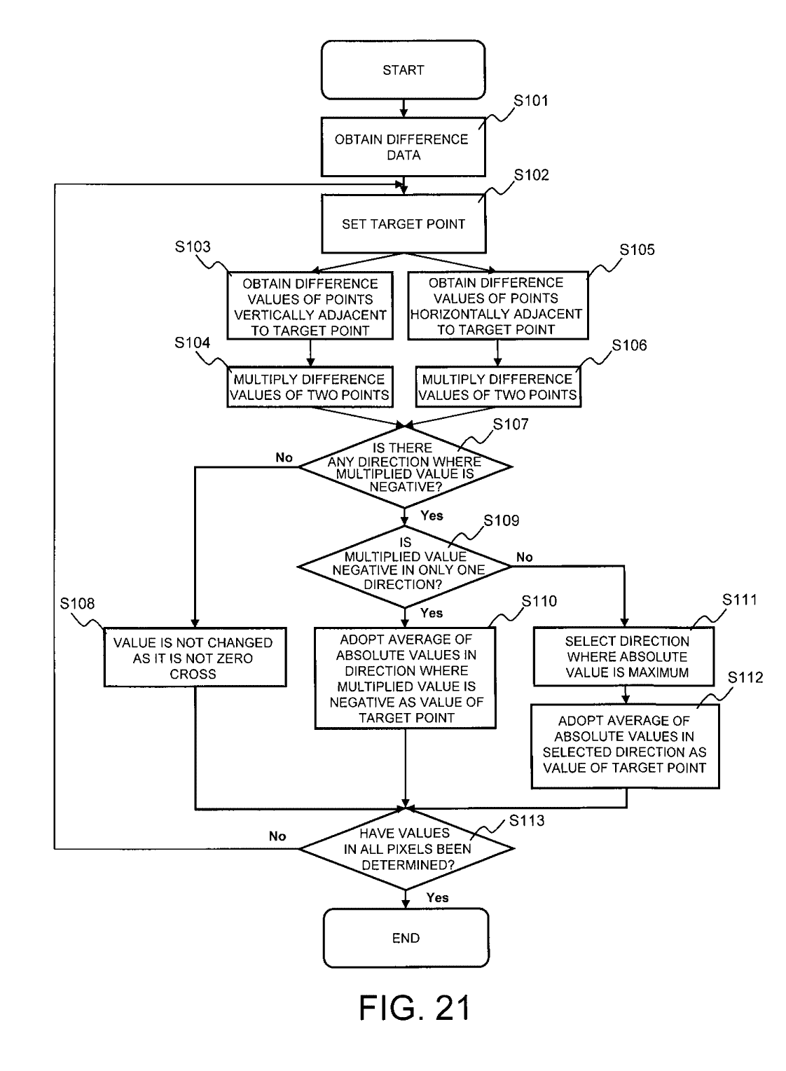

FIG. 21 shows a diagram of a specific example of processing performed in the zero cross removal section 5101 (FIG. 20). In S101, difference data are obtained from the subtracter 13-3. In S102, a target point is set. In S103, difference values of points vertically adjacent to the target point (the y-axis direction in the image) are obtained. In S104, the obtained difference values of the two points are multiplied. In S105, difference values of points horizontally adjacent to the target point (the x-axis direction in the image) are obtained. In S106, the obtained difference values of the two points are multiplied.

In S107, determination is made as to whether or not at least one of the multiplied values obtained in S104 and S106 is negative. The process proceeds to S109 if even only one value is negative, or if not, the process proceeds to S108.

In S108, determination that the target point is not zero cross is made, and the process proceeds to S113 without changing the difference value of the target point (pixel).

In S109, determination is made as to whether only one of the multiplied values obtained in S104 and S106 is negative. If only one of the multiplied values is negative, the process proceeds to S110, and if the two multiplied values are both negative, the process proceeds to S111. In S110, an average between absolute values of the two points in the direction where the multiplied value becomes negative is assumed as a value of the target point, and the process proceeds to S113.

In S111, by selecting a direction in which absolute values of the multiplied values obtained in S104 and S106 are maximum, a maximum inclination direction is selected, and the process proceeds to S112. In S112, an average between the absolute values of the two points in the direction selected in S111 is adopted as a value of the target point, and the process proceeds to S113.

In S113, determination is made as to whether or not values of all the target points have been determined. If the values of all the target points are determined, the process is ended, and if not, the process returns to S102, and processing for the next target point is performed.

Although, in the above-described specific example, the difference values of the points adjacent to the target point vertically and horizontally have been obtained, this is not limiting, and, for example, a step of calculating a difference value in the orthogonal direction may be provided to detect zero cross in more directions. Further, although, in the above-described specific example, the comparison has been made for each direction, the maximum inclination direction may be calculated by obtaining all values of adjacent points and performing, for example, principal component analysis. Although, in zero cross removal, preferably, the average of the absolute values in the maximum inclination direction is input, this is not limiting, and for example, an average value of absolute values of adjacent four points may also be input.

Referring to FIG. 15 again, the Str² component obtained in the structure calculation section 51 is input to the data update section 61. The data update section 61 updates a multiGₙ buffer and a multiStr buffer used in estimation of motion of the tissue and estimation of the structure of the tissue.

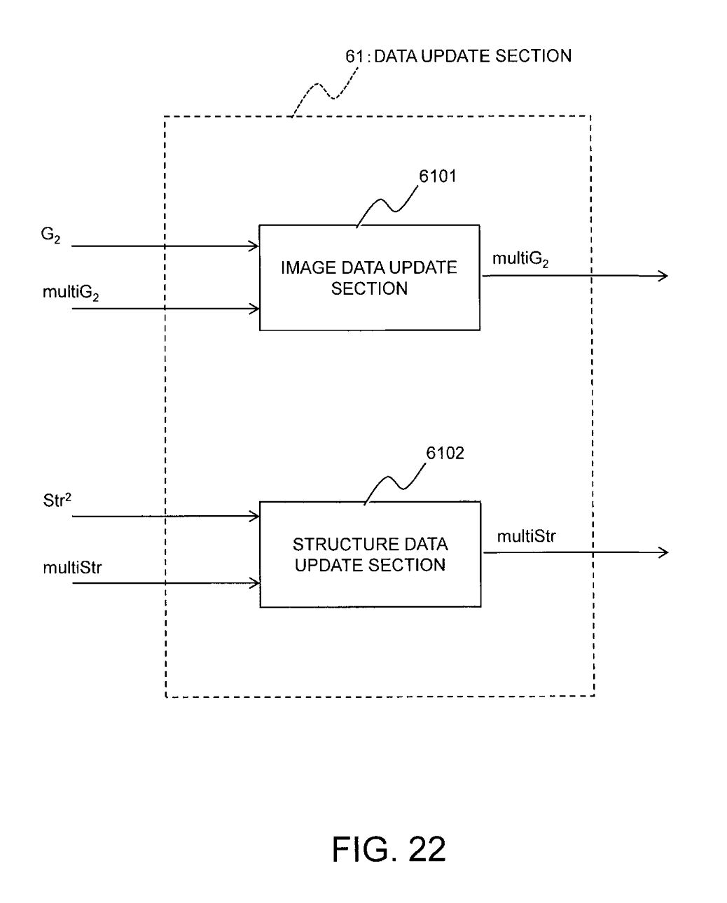

FIG. 22 shows a diagram of an internal structure of the data update section 61 (FIG. 15). The data update section 61 has the structure as shown in the figure. More specifically, the data update section 61 is composed of an image data update section 6101 that updates the multiGₙ buffer for storing the Gₙ component (image data) obtained before the current frame, using more than one data of the Gₙ component of the current frame generated in the multiresolution decomposition section 31 (FIG. 15), and a structure data update section 6102 that updates the multiStr buffer for storing the structure data obtained before the current frame, using the structure estimation value Str² of the current frame generated in the structure calculation section 51 (FIG. 15). Although, in the specific example in FIG. 22, it is assumed that n=2, this is not limiting, and for example, a greater number of layers may be updated.

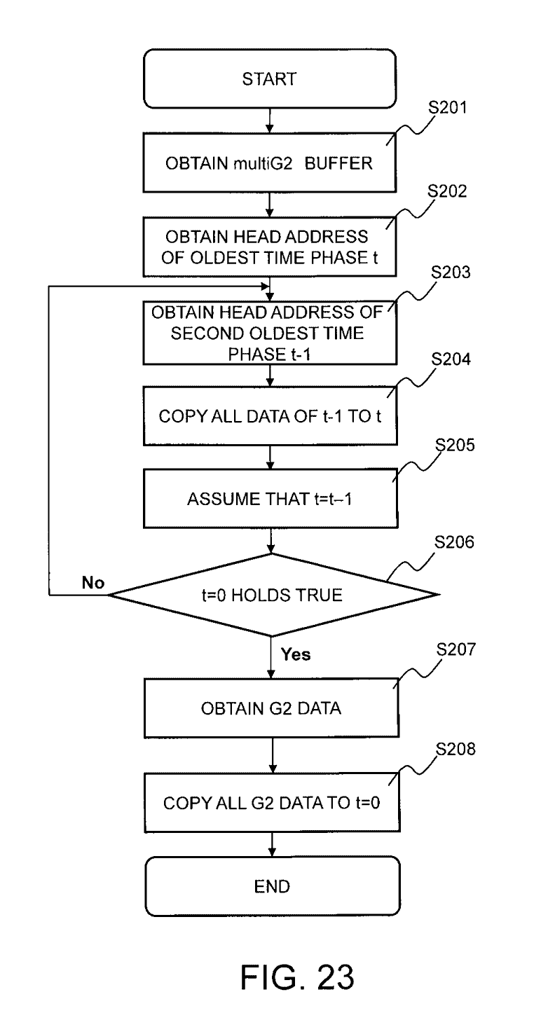

FIG. 23 shows a diagram of a specific example of processing performed in the data update section 61 (FIG. 22). FIG. 23 shows a flowchart of processing performed in the image data update section 6101 (FIG. 22).

In S201, a multiG₂ buffer is obtained. In S202, a head address of the oldest time phase t is obtained. In S203, a head address of the second oldest time phase t-1 is obtained. In S204, a data array of the time phase t-1 is all copied to a data array of the time phase t. In S205, it is assumed that t=t-1.

In S206, determination is made as to whether or not t=0 holds true. If t=0 holds true, the process proceeds to S207, and if not, the process proceeds to S203 to copy the next time phase. In S207, the G₂ component of the current frame is obtained. In S208, the G₂ component of the data of the current time phase is copied to the data array of t=0, and updating of the multiG₂ buffer is ended.

According to the specific example shown in FIG. 23, the image data update section 6101 outputs the multiG₂ buffer composed of the G₂ components of the three frames.

Further, by adopting Str² as G₂ in the specific example of FIG. 23, the structure data update section 61 also outputs the multiStr buffer composed of the Str² components of the three frames through processing similar to that of the flowchart in FIG. 23. The above-described updating method does not necessarily have to be adopted, and, for example, processing of switching pointers may also be adopted.

Referring to FIG. 15 again, the multiGₙ buffer and the multiStr buffer updated in the data update section 61 are input to the background subtraction section 71. The multiGₙ buffer and the multiStr buffer are further input to the data update section 61 again for the next frame calculation.

The background subtraction section 71 calculates a fogging component included in the Gₙ component based on the estimation of the tissue motion and the estimation of the tissue structure, thereby calculating an nrGₙ component subjected to fogging reduction processing.

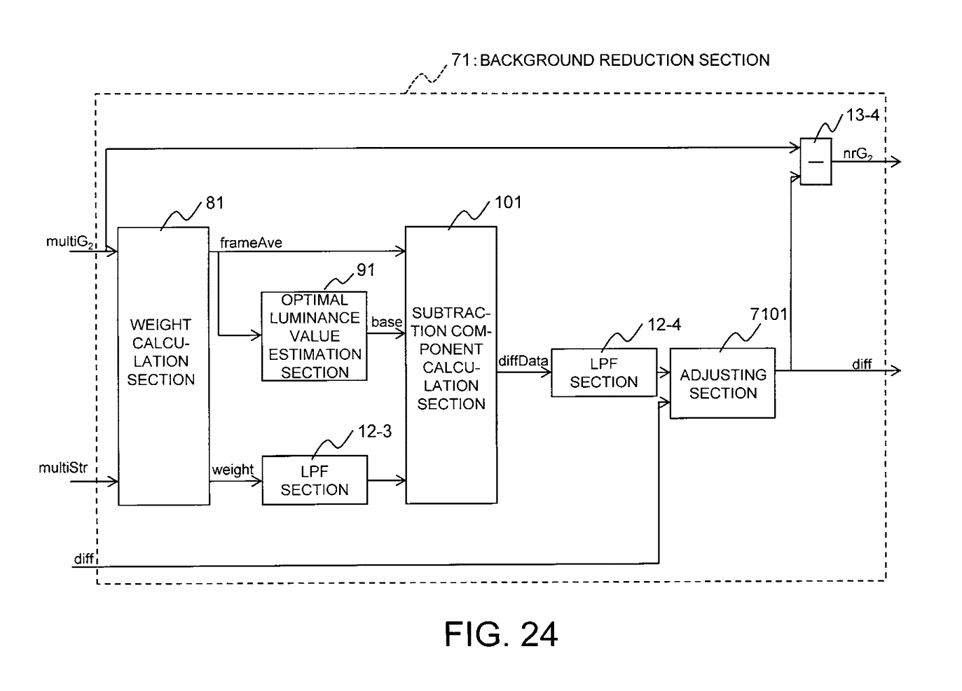

FIG. 24 shows a diagram of an internal structure of the background subtraction section 71 (FIG. 15). The background subtraction section 71 has the structure as shown in the figure. A weight calculation section 81 calculates an average image frameAve component and a subtraction weight weight component based on the multiGₙ buffer and the multiStr buffer. An optimal luminance value estimation section 91 calculates an optimal luminance value base of the current frame.

A subtraction component calculation section 101 calculates a subtraction component from the average image frameAve component calculated in the weight calculation section 81, the optimal luminance value base calculated in the optical luminance value estimation section 91, and the subtraction weight weight calculated in the weight calculation section 81 and subjected to a low-pass filter (LPF) in an LPF section 12-3.

Preferably, the calculated subtraction component is subjected to a low-pass filter (LPF) in an LPF section 12-4 and smoothed in the space direction, and then, it is smoothed in the time direction in an adjusting section 7101 based on the following equation.

diff_{i,j}^{t}=diffData_{i,j}×beta+diff_{i,j}^{t-1}×(1−beta) [Equation 1]

diff: subtraction components up to the last frame

diffData: a calcula: a calculated subtraction

beta: parameter

In doing so, the diagnostic image reconstructed by a below-described processing can suppress local subtraction and a large luminance change in the same pixel between the frames and provide a diagnostic image with less sense of congruity. The subtracter 13-4 subtracts the spatially and timely-smoothed subtraction component from the Gₙ component of the current frame stored in the multiGₙ component, to thereby calculate an nrGₙ component from which the fogging is reduced.

Although, in the above-described specific example, it is assumed that n=2, this specific example is not limiting. Further, although, in the above-described specific example, calculation has been carried out based on the subtraction component calculated in the current frame in the adjusting section 7101 and the weighting addition value of the subtraction component that has been updated up to the last frame, all the data so far or similar parameters may also be stored to perform weighting as appropriate.

The nrGₙ component obtained in the background subtraction section 71 is input to the image reconstruction section 111. The subtraction component obtained in the background subtraction section 71 is further input to the background subtraction section 71 again for the next frame calculation. The weighting calculation section 81 calculates the average image frameAve component and the subtraction weight weight as an evaluation value representing an estimation value of fogging.

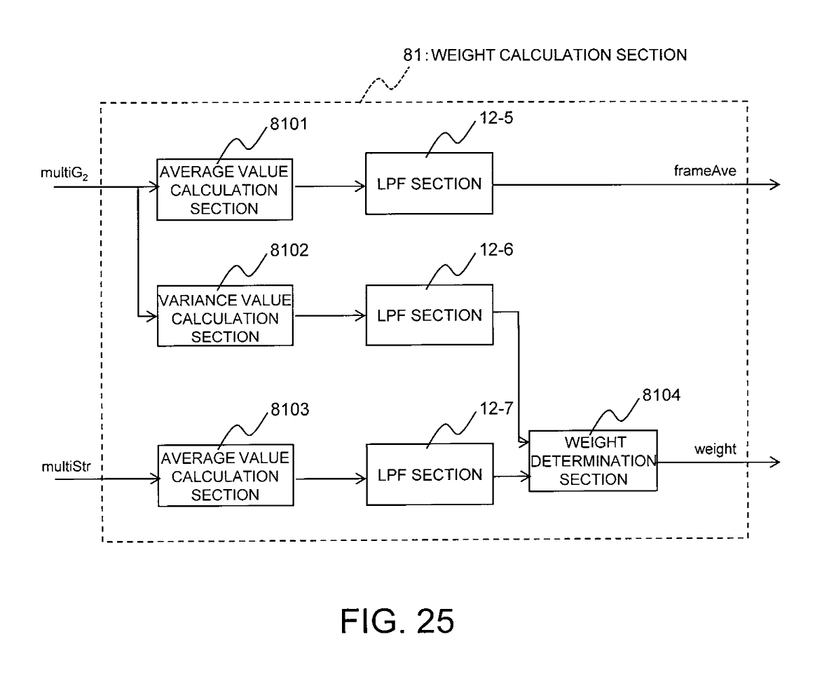

FIG. 25 shows a diagram of an internal structure of the weight calculation section 81 (FIG. 24). The weight calculation section 81 has the structure as shown in the figure. An average value calculation section 8101 calculates, for each pixel, luminance values of at least one G₂ component stored in the input multiG₂ buffer or average values of a plurality of G₂ components. A variance value calculation section 8102 calculates, for each pixel, variance values of the luminance values of the plurality of G₂ components stored in the multiG₂ buffer. An average value calculation section 8103 calculates, for each pixel, strength values of at least one Str² component stored in the input multiStr buffer or average values of a plurality of Str² components.

The values calculated in the average value calculation section 8101, the variance value calculation section 8102, and the average value calculation section 8103 are subjected to the low-pass filters in LPF sections 12-5, 12-6, and 12-7, respectively. The data subjected to the low-pass filter in the LPF section 12-5 are output as the average image frameAve. The data subjected to the low-pass filter in the LPF sections 12-6 and 12-7 are also input to a weight determination section 8104.

Here, calculation performed in the weight determination section 8104 will be described in more detail. The weight determination section 8104 calculates a weight weight that maintains, among the subtraction candidate components obtained after processing described below, components that are estimated to be the fogging and removes components that are not estimated to be the fogging, so as not to allow them to be the subtraction components. In other words, as an evaluation value representing the estimation value of the fogging, the subtraction weight weight is given as 0≦weight≦1. This is a normalized evaluation value indicating the “conspicuity” of the fogging, and, in order to calculate this evaluation value, in the present embodiment, an evaluation value of the fogging is obtained using the motion and the structure as examples.

The fogging is noise that appears near the probe 10 and has a small amount of motion and no structure. Therefore, preferably, a component with a smaller amount of motion and weaker structure strength is determined to be fogging, and the weight is closer to 1. In contrast, if the component has a larger amount of motion or a stronger structure, the component may have information of the myocardium and the like, and the weight is closer to 0.

In doing so, the weight determination section 8104 calculates the subtraction weight weight based on the values calculated in the LPF sections 12-6 and 12-7, for example, using a method described below.

First, because the value calculated in the LPF section 12-6 is a value obtained by smoothing, for each pixel, the variance values using the plurality of frames, if this value is small, it is understood that, in that region, the luminance change was small, and the motion of the pixel was small. Thus, the weight for the motion can be calculated, for example, according to a reduction function in the following equation using a calculated value in the pixel (i, j) and a parameter gamma.

$${weight}_{i , j}^{move} = exp ( - \frac{{{σ}^{- 2}}_{i , j}}{2 \cdot {gamma}^{2}} ) \\ 0 \leq {weight}_{i , j}^{move} \leq 1 \qquad [ Equation 2 ]$$

Calculated value σ̅_{i,j}² in the pixel (i, j)

Next, because the value calculated in the LPF section 12-7 is a value obtained by smoothing, for each pixel, the structure estimation values using the plurality of frames, if this value is small, it is understood that, in that region, the structure is weak. Thus, the weight for the structure can be calculated, for example, according to a reduction function in the following equation using the calculated value in the pixel (i, j) and a parameter delta.

$${weight}_{i , j}^{str} = exp ( - \frac{{{str}^{- 2}}_{i , j}}{2 \cdot {delta}^{2}} ) \\ 0 \leq {weight}_{i , j}^{str} \leq 1 \qquad [ Equation 3 ]$$

Calculated value s̅t̅r̅_{i,j}² in the pixel (i, j)

The weighting for the subtraction candidate component can be calculated, for example, according to a reduction function in the following equation using the weight for the motion in Equation 2 and the weight for the structure in Equation 3.

$${weight}_{i , j} = {weight}_{i , j}^{move} \times {weight}_{i , j}^{str} = exp ( - \frac{{{σ}^{- 2}}_{i , j}}{2 \cdot {gamma}^{2}} ) \times exp ( - \frac{{{str}^{- 2}}_{i , j}}{2 \cdot {delta}^{2}} ) \qquad [ Equation 4 ]$$

Although the above-described specific example has used the reduction function in which the weight becomes closer to 1 in the spot which is estimated to be fogging, a reduction function other than this specific example may also be used. In addition, although, in the present embodiment, the variance value calculation section 8102 and the average value calculation section 8103 have performed, for each pixel, calculations based on the values of the plurality of frames, calculation may be performed using pixel data in the range given by the kernel size m*n (m≧0, n≧0), for example. Further, although, in the present embodiment, weighting for the motion has been calculated from the variance of the luminance value, weighting may also be calculated using an evaluation value used in performing block matching, etc., such as, for example, an SAD (Sum of Absolute Difference).

Referring to FIG. 24 again, the average image frameAve obtained in the weight calculation section 81 is output to the optimal luminance value estimation section 91 and the subtraction component calculation section 101. Further, the subtraction weight weight obtained in the weight calculation section 81 is subjected to the LPF processing in the LPF section 12-3 and then output to the subtraction component calculation section 101. The optimal luminance value estimation section 91 estimates an optimal luminance value base of the input data.

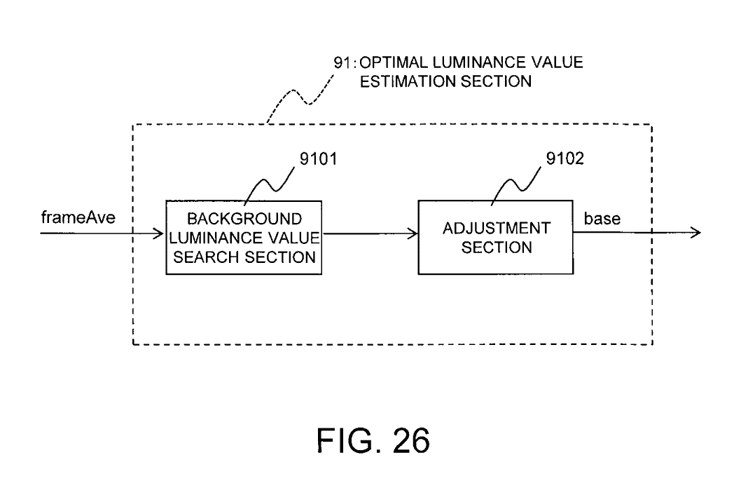

FIG. 26 shows a diagram of an internal structure of the optimal luminance value estimation section 91 (FIG. 24). The optimal luminance value estimation section 91 has the structure as shown in the figure. A background luminance value search section 9101 searches a minimum value of the input data; that is, an optimal luminance value min in the average image frameAve, and an adjustment section 9102 makes an adjustment using a parameter epsilon, for example. In other words, the optimal luminance value estimation section 91 performs calculation indicated in the following equation, thereby calculating the optical luminance value base.

base=min(frameAve)×epsilon [Equation 5]

Although, in the present embodiment, the optical luminance value base has been calculated by the above-described method, this is not limiting. Preferably, because the optimal luminance value base is a value for estimating the luminance value which should be held by a noise portion such as fogging and the like, an optional luminance value other than the optimal luminance value may be automatically calculated from the histogram of the image using a discrimination analysis method. Further, an optional luminance value may be given by the user.

As such, by estimating the optimal luminance value, it is possible to control the subtraction candidate component obtained through a below-described processing, and make an adjustment so as not to excessively reduce the luminance of the portion estimated to be the fogging. In doing so, it is possible to prevent the diagnostic image reconstructed through the below-described processing from including a sense of congruity.

Referring to FIG. 24 again, the optimal luminance value base calculated in the optimal luminance value estimation section 91 is output to the subtraction component calculation section 101. The subtraction component calculation section 101 estimates a fogging component of the target frame and outputs the component as the subtraction component.

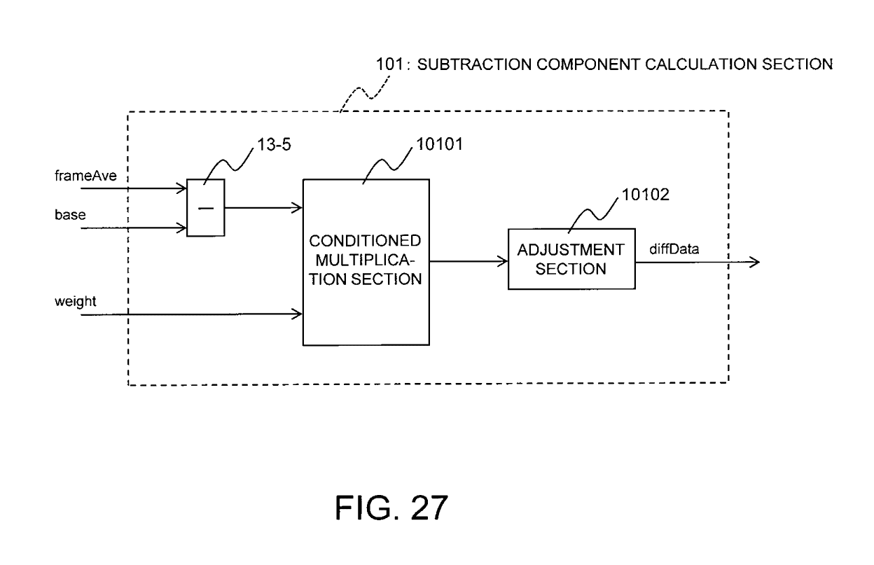

FIG. 27 shows a diagram of an internal structure of the subtraction component calculation section 101 (FIG. 24). The subtraction component calculation section 101 has the structure as shown in the figure. A subtracter 13-5 subtracts the optimal luminance value base calculated in the optimal luminance value estimation section 91 (FIG. 24) from the average image frameAve calculated in the weight calculation section 81 (FIG. 24), thereby calculating a subtraction candidate component.

A conditioned multiplication section 10101 calculates a subtraction component diffData from the calculated subtraction candidate component and the subtraction weight weight. An adjustment section 10102 makes an adjustment of the obtained subtraction component diffData using a parameter alpha, for example. The subtraction component calculation section 101 calculates, for each pixel (i, j), the subtraction component diffData based on the following equation, for example.

diffData_{i,j}=alpha×(frameAve_{i,j}−base)×weight_{i,j} [Equation 6]

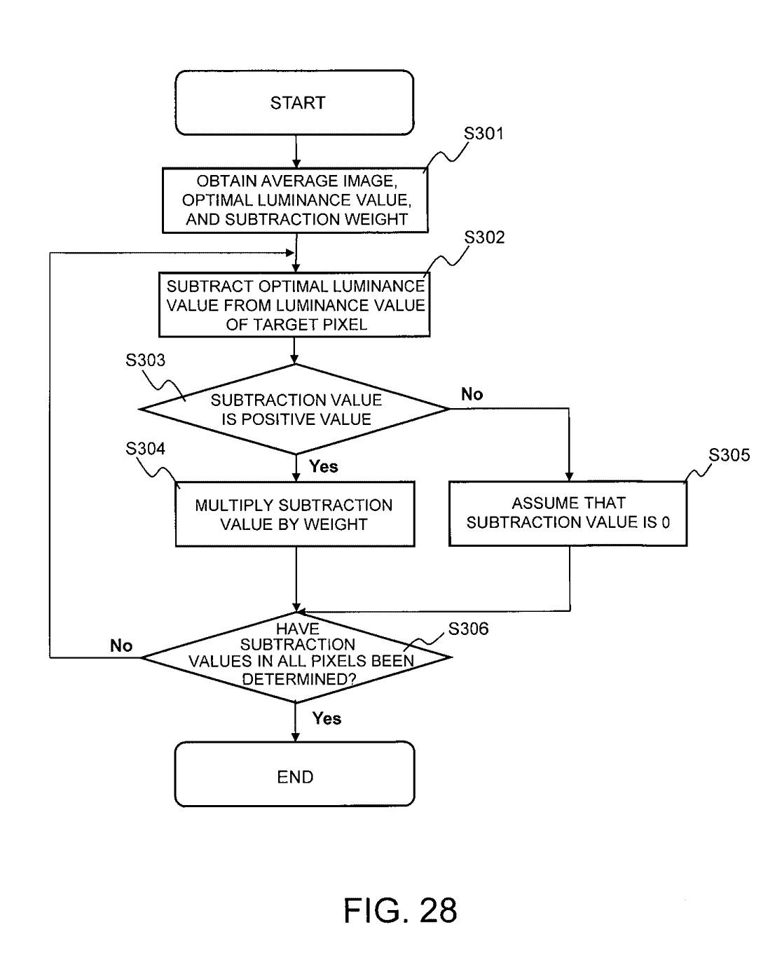

FIG. 28 shows a diagram of a specific example of processing performed in the conditioned multiplication section 10101 (FIG. 27). FIG. 28 shows a flowchart of processing performed in the conditioned multiplication section 10101. In S301, the average image frameAve, the optimal luminance value base, and the subtraction weight weight are obtained. In S302, a target pixel is set, and the optimal luminance value base is subtracted from the luminance value of the target pixel, thereby calculating a subtraction candidate component. In S303, determination is made as to whether or not the subtraction candidate component is positive. If the subtraction candidate component is positive, the process proceeds to S304, while if the component is negative, the process proceeds to S305.

In S304, because the subtraction candidate component is positive, the component is multiplied by the subtraction weight weight, thereby determining a subtracted value. In S305, because the subtraction candidate component is negative, the pixel has a luminance value lower than the optimal luminance value. Then, the subtracted value is set to be 0 so as not to perform processing. In S306, determination is made as to whether or not values of all the target pixels have been determined. If the values of all the target pixels have been determined, the process is ended, and if not, the process proceeds to S302 to determine a value of the next target pixel.

Referring to FIG. 24 again, the subtraction component diffData obtained in the subtraction component calculation section 101 is subjected to the LPF processing in the LPF section 12-4 and output to the adjusting section 7101. As described above, the adjusting section 7101 performs processing based on Equation 1. Thus, an nrGₙ component from which the fogging is reduced is output from the background subtraction section 71 to the image reconstruction section 111 (FIG. 15). The image reconstruction section 111 performs reconstruction processing of the Gaussian pyramid using the nrGₙ component from which the fogging component is reduced, the L₀ component, and the L₁ component.

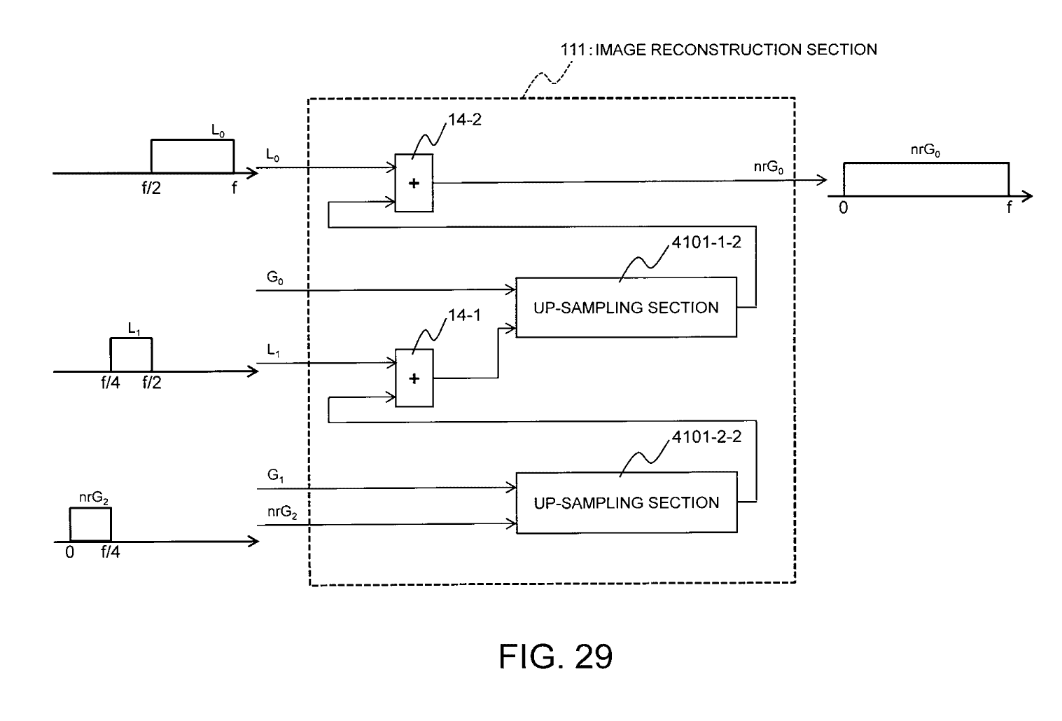

FIG. 29 shows a diagram of an internal structure of the image reconstruction section 111 (FIG. 15). The image reconstruction section 111 has the structure as shown in the figure. The input nrGₙ component is input to up-sampling sections 4101-1-2 and 4101-2-2, thereby being subjected to the up-sampling processing. The up-sampled component is input to adders 14-1 and 14-2 together with the Lₙ components, thereby being subjected to addition processing.

Thus, there are obtained image data nrG₀ from which the fogging has been reduced, and preferably, from which the fogging has been removed. The image data nrG₀ have sample density and the resolution equal to those of the image data input to the image processing section 20.

FIG. 29 shows a specific example of a frequency band of each component. nrG₂ is a component obtained based on G₂ (FIG. 16) and has a frequency hand of 0 to f/4. Further, L₀ has a frequency band of f/2 to f, and L₁ has a frequency band of f/4 to f/2 (FIG. 18). Then, nrG₀ has a frequency band of 0 to f, because it is obtained by adding nrG₂, L₁, and L₀. That is, nrG₀ reconstructed in the image reconstruction section 111 has the same frequency band as the diagnostic image G₀, which is the original image.

Further, although in the above-described embodiment, the G₀ component, the G₁ component, the L₀ component, the L₁ component, and the nrG₂ component from which the fogging is reduced have been obtained, this is not limiting, and more layers may be used. Furthermore, in the above embodiment, preferably, by performing the fogging reduction processing in the Gₙ component of the layer where n≧1, and adding an Lₖ component (0≦k≦n) while up-sampling the nrGₙ component from which the fogging is reduced, it is possible to reduce the “stickiness” observed in a simple filter and the like and perform reconstruction of the diagnostic image with less sense of congruity.

The image data nrG₀ reconstructed in the image reconstruction section 111 are transmitted to the display processing section 30, and this allows the display section 40 to display an ultrasonic image from which the fogging is reduced, and more preferably, an ultrasonic from which the fogging is removed. Thus, for example, by reducing the fogging efficiently without reducing the myocardium information to a large extent, it is possible to display the ultrasonic image having a good visibility (for example, a B-mode image).

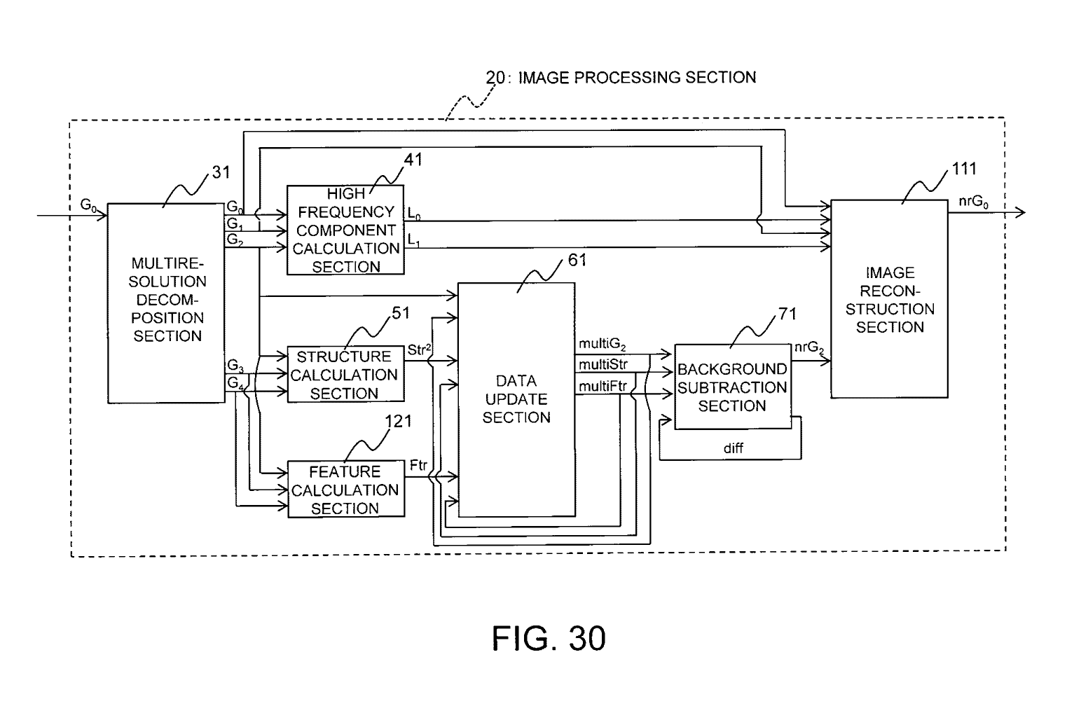

FIG. 30 shows a diagram of a variance (second embodiment) of the image processing section 20. It differs from the image processing section 20 in FIG. 15 in that it additionally has a feature calculation section 121 in FIG. 30 and uses, in addition to the two features of the motion of the tissue and the structure of the tissue, third or more features in estimation of the fogging. Here, the third or more features calculated in the feature calculation section 121 include, for example, a direction of the tissue, a difference between the frame directions, color information of the image, etc.

Although, in the specific example shown in FIG. 30, in order to specify the processing, it is assumed that n=2, and a feature estimation value Ftr is obtained using the G₂ component to the G₄ component, this is not limiting, and more than one data of the generated Gₙ components may be input, to thereby calculate the feature estimation value Ftr.

Further, although in the above-described variation (second embodiment), the structure where the only one feature calculation section 121 is positioned in the image processing section 20 has been indicated, this is not limiting, and the number of feature calculation section 121 may be increased according to three or more number of features desired to be used.

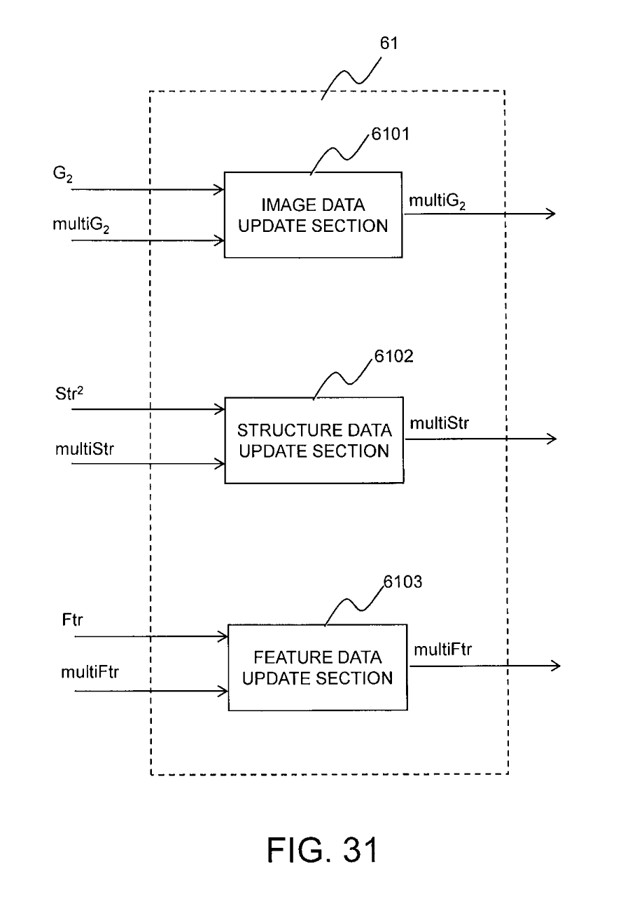

FIG. 31 shows a diagram of an internal structure of the data update section 61 (FIG. 30). The data update section 61 has the structure as shown in the figure. It differs from the data update section 61 in FIG. 22 in that, in FIG. 31, it has a feature data update section 6103, in addition to the image data update section 6101 and the structure data update section 6102. The feature data update section 6103 updates a multiFtr buffer that stores the structure data obtained before the current frame, using the feature estimation value Ftr of the current frame generated in the feature calculation section 121 (FIG. 30).

Although, in this variation (second embodiment), the structure where the only one feature data update section 6103 is positioned in the data update section 61 has been indicated, this is not limiting, and the number of the feature data update section 6103 may be increased according to three or more number of features desired to be used.

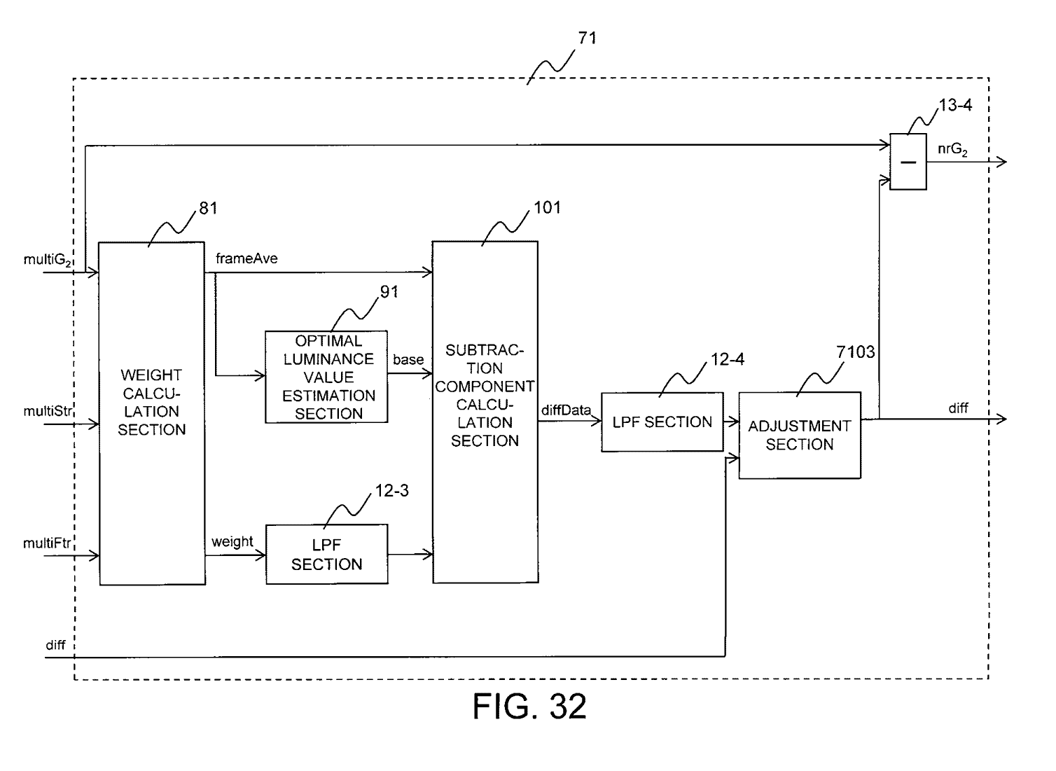

FIG. 32 shows a diagram of an internal structure of the background subtraction section 71 (FIG. 30). The background subtraction section 71 has the structure as shown in the figure. It differs from the background subtraction section 71 in FIG. 24 in that, in FIG. 32, as an input, the multiFtr buffer is added, and that the multiFtr buffer is input to the weight calculation section 81.

Although, in this variation (second embodiment), the structure where one multiFtr buffer is added as an input to the background subtraction section 71 has been indicated, this is not limiting, and the number of input buffers may be increased according to three or more number of features desired to be used. Further, in conjunction with this, the number of buffers input to the weight calculation section 81 may be increased according to the number of features desired to be used.

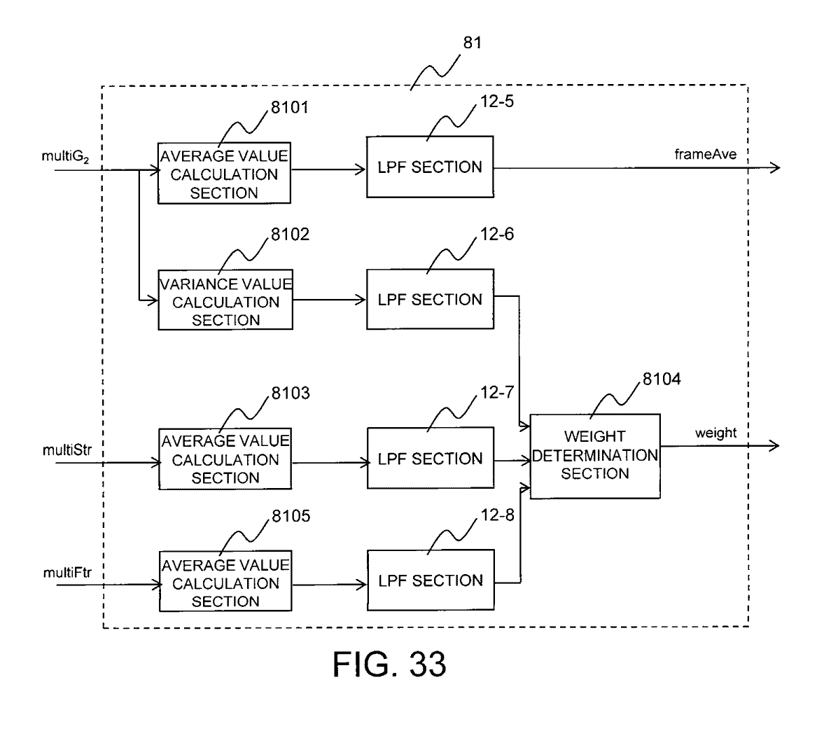

FIG. 33 shows a diagram of an internal structure of the weight calculation section 81 (FIG. 32). The weight calculation section 81 has the structure as shown in the figure. It differs from the weight calculation section 81 in FIG. 25 in that, in FIG. 33, the multiFtr buffer is added as an input; an average value calculation section 8105 is added to calculate, for each pixel, a luminance value of at least one Ftr component stored in the multiFtr buffer or an average value of a plurality of Ftr components; and that the calculated value is subjected to a low-pass filter (LPF) in an LPF section 12-8 which is also added, and input to the weight determination section 8104.

Although, in this variation (second embodiment), the structure where one multiFtr buffer is added as an input in the weight calculation section 81 has been indicated, this is not limiting, and the number of input buffers may be increased according to three or more number of features desired to be used. In addition, in conjunction with this, the number of the average value calculation section 8105 and the LPF section 12-8 may be increased according to three or more number of features desired to be used.

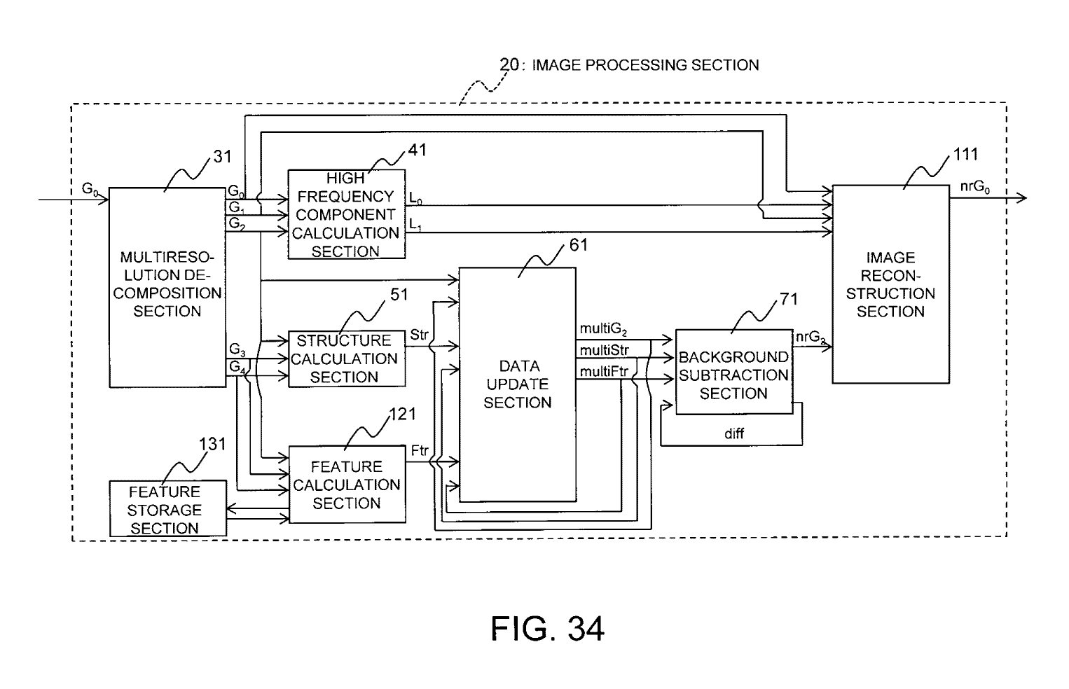

FIG. 34 shows a diagram of another variation (third embodiment) of the image processing section 20. It differs from the image processing section 20 in FIGS. 15 and 30 in that the image processing section in FIG. 34 adopts a feature obtained by machine learning, etc. in advance. In conjunction with this, in addition to the structure shown in the second embodiment in FIG. 30, a feature storage section 131 is added in FIG. 34.

For example, the feature storage section 131 stores in advance a feature of the fogging portion and a return value according to that feature. At this time, there may also be stored a feature of the structure of the myocardium, etc. that is important for diagnosis and a return value according to that feature. Therefore, by inputting the features calculated in the feature calculation section 121 to the feature storage section 131, the feature calculation section 121 can obtain return values according to the features. Features are calculated by using these return values as the feature estimation values Ftr.

Although, in this variation (third embodiment), the structure where the only one feature storage section 131 is positioned in the image processing section 20 has been indicated, this is not limiting, and the number of feature storage section 131 may be increased to three or more number of features desired to be used. Further, the second embodiment and the third embodiment can be used together.

Although the image processing based on the two-dimensional image has been described above, fogging reduction processing for a three-dimensional image may also be performed. In case of processing of a three-dimensional image, preferably, the down-sampling section 3101 (FIG. 16) and the up-sampling section 4101 (FIGS. 18, 20, and 29), which have applied the two-dimensional low-pass filters, are changed to apply three-dimensional low-pass filters. However, for example, a one-dimensional low-pass filter may be applied in each direction in three dimensions, or, alternatively, a two-dimensional low-pass filter is applied to a cross section including any two directions, and then a one-dimensional low-pass filter may be applied to the remaining one direction.

Further, the signals obtained from the transmitting and receiving section 12 may be subjected to processing, such as wave detection and logarithmic transformation, and subsequently subjected to fogging reduction in the image processing section 20. Subsequently, the coordinate transform processing may be performed in a digital scan converter. Naturally, the signals obtained from the transmitting and receiving section 12 may be subjected to fogging reduction in the image processing section 20 and subsequently subjected to the processing, such as wave detection and logarithmic transformation. Alternatively, the signals may be subjected to the coordinate transform processing in the digital scan converter and then subjected to fogging reduction in the image processing section 20.

Although the preferred embodiments of the present invention have been described, the above-described embodiments are merely examples in all respects and are not intended to limit the scope of the present invention. The present invention includes various variations without departing from the spirit of the present invention.

### REFERENCE SIGNS LIST

10 Probe
12 Transmitting and receiving section
20 Image processing section
30 Image processing section
40 Display section

1. An ultrasonic diagnostic device comprising:
a probe that transmits and receives ultrasonic waves;
a transmitting and receiving section that obtains a received signal from the probe by controlling the probe;
a resolution processing section that forms a plurality of resolution images, each having different resolution, by resolution conversion processing of an ultrasonic image obtained based on the received signal;
a reduction processing section that determines the degree of reduction in each portion of the image based on the plurality of resolution images; and
an image forming section that forms an ultrasonic image subjected to reduction processing according to the degree of reduction in each portion of the image.

2. The ultrasonic diagnostic device according to claim 1, wherein the reduction processing section estimates the degree of structure for each portion in the image based on a difference image between the plurality of resolution images, and determines the degree of reduction for each portion in the image based on the estimation result.

3. The ultrasonic diagnostic device according to claim 1, wherein the reduction processing section estimates the degree of motion for each portion in the image based on images obtained over a plurality of time phases from at least one of the plurality of resolution images, and determines the degree of reduction for each portion in the image based on the estimation result.

4. The ultrasonic diagnostic device according to claim 2, wherein the reduction processing section estimates the degree of motion for each portion in the image based on images obtained over a plurality of time phases from at least one of the plurality of resolution images, and determines the degree of reduction for each portion in the image based on the estimation result of the degree of motion and the estimation result of the degree of structure.

5. The ultrasonic diagnostic device according to claim 1, wherein:
the reduction processing section estimates the degree of structure and the degree of motion for each portion in the image, and determines a subtraction component that determines the degree of reduction for each portion in the image based on the degree of tissue and the degree of motion; and
the image forming section forms an ultrasonic image from which the subtraction component is subtracted.

6. The ultrasonic diagnostic device according to claim 5, wherein the reduction processing section estimates the degree of structure for each portion in the image based on a difference image between the plurality of resolution images.

7. The ultrasonic diagnostic device according to claim 5, wherein
the reduction processing section estimates the degree of motion for each portion in the image based on images obtained over a plurality of time phases from at least one of the plurality of resolution images.

8. The ultrasonic diagnostic device according to claim 5, wherein the reduction processing section subtracts an optimal luminance value determined based on a lowest luminance value in the ultrasonic image from a luminance value of each pixel, thereby generating a subtraction candidate component, and determines the subtraction component based on a subtraction weight and the subtraction candidate component, the subtraction weight being determined according to the degree of structure and the degree of motion.

9. The ultrasonic diagnostic device according to claim 8, wherein the reduction processing section estimates the degree of structure for each portion in the image based on a difference image between the plurality of resolution images.

10. The ultrasonic diagnostic device according to claim 8, wherein the reduction processing section estimates the degree of motion for each portion in the image based on images obtained over a plurality of time phases from at least one of the plurality of resolution images.

11. The ultrasonic diagnostic device according to claim 1, wherein:
the resolution processing section forms, as the plurality of resolution images, at least one high-resolution image and a plurality of low-resolution images;
the reduction processing section determines the degree of reduction in each portion in the image based on the plurality of low-resolution images, and forms a low-resolution image component that has been subjected to reduction processing according to the degree of reduction; and
the image forming section synthesizes a high-resolution image component obtained from the high-resolution image and the low-resolution image component, thereby forming an ultrasonic image.

12. The ultrasonic diagnostic device according to claim 11, wherein the reduction processing section estimates the degree of structure for each portion in the image based on a difference image between the plurality of low-resolution images, and determines the degree of reduction for each portion in the image based on the estimation result.

13. The ultrasonic diagnostic device according to claim 11, wherein the reduction processing section estimates the degree of motion for each portion in the image based on images obtained over a plurality of time phases from at least one of the plurality of low-resolution images, and determines the degree of reduction for each portion in the image based on the estimation result.
